(19) United States
(12) Patent Application Publication
(10) Pub. No.: US 2022/0183999 A1
(43) Pub. Date: Jun. 16, 2022

(54) Cannabidiol Pharmaceutical Compositions

(71) Applicant: Sorrento Therapeutics, Inc., San Diego, CA (US)

(72) Inventors: Hui Xie, San Diego, CA (US); Hua Wang, San Diego, CA (US); Henry Hongjun Ji, Rancho Santa Fe, CA (US); Jonathan D. Wang, San Diego, CA (US)

(73) Assignee: Sorrento Therapeutics, Inc., San Diego, CA (US)

(21) Appl. No.: 17/601,357

(22) PCT Filed: Apr. 3, 2020
(86) PCT No.: PCT/US2020/026585

§ 371 Date: Oct. 4, 2021

Related U.S. Application Data
(60) Provisional application No. 62/830,352, filed on Apr. 5, 2019.

(51) Int. Cl. A61K 31/05; A61K 47/10; A61K 47/26; A61K 47/44; A61K 9/08; A61K 9/00

(52) U.S. Cl. CPC A61K 31/05; A61K 47/10; A61K 9/0095; A61K 47/44; A61K 9/08; A61K 47/26

(57) ABSTRACT

Disclosed herein are aqueous pharmaceutical compositions comprising cannabidiol, method of making such compositions, and methods of treatment using such pharmaceutical compositions.

This application claims the benefit of priority of U.S. Provisional Patent Application No. 62/830,352, filed Apr. 5, 2019, which is incorporated by reference herein in its entirety.

The present disclosure provides pharmaceutical compositions comprising cannabidiol and methods of treating pain or a neurological disorder comprising administering such pharmaceutical compositions.

### I. INTRODUCTION AND SUMMARY

Cannabidiol (CBD) is a phytocannabinoid component of the Cannabis plant that was first isolated in 1940 (Boggs et al., Neuropsychopharmacology 2017, 43(1), 142-154). CBD has the following structure:

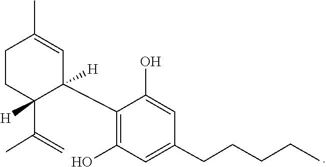 [SMILES: C=C(C)[C@@H]1CCC(C)=C[C@H]1c1c(O)cc(CCCCC)cc1O]

CBD has been shown to act as an indirect antagonist of CB₁ and CB₂ receptors, an antagonist of GPR55 (a G protein-coupled receptor that is expressed in the brain), and inverse agonist of GPR3, GPR6, and GPR12, a partial agonist of serotonin 5-HT_{1A} receptor, and an allosteric modulator of the μ and δ-opioid receptors. CBD has been studied as a treatment to ameliorate one or more symptoms for a range of disorders, including pain (such as neuropathic pain or cancer-related pain), spasticity, anxiety, cognition, movement disorders, epilepsy (including childhood epilepsy, such as Lennox-Gastaut syndrome or Dravet syndrome, and dementia (including Alzheimer's disease, Vascular Dementia, Dementia with Lewy bodies (DLB), Parkinson's disease, Frontotemporal dementia and Huntington's disease).

CBD is FDA-approved in the United States as EPIDIOLEX® (oral solution, 100 mg/mL) for treatment of seizures associated with Lennox-Gastaut syndrome or Dravet syndrome and in the United Kingdom and other countries under the name SATIVEX® (1:1 mixture of CBD and delta 9-tetrahydrocannabinol (THC)) for treatment of spasticity due to multiple sclerosis.

CBD is nearly insoluble in water (0.013 mg/mL). Drug substances with poor aqueous solubility present problems in the context of drug delivery. The aqueous solubility of a pharmaceutical agent impacts various pharmacokinetic and pharmacodynamic properties, such as absorption, distribution, Tₘₐₓ, Cₘₐₓ, and clearance. Such compounds often exhibit low and/or variable absorption and bioavailability. Poor pharmacodynamic properties limit the therapeutic effect and treatment flexibility for an agent.

Poor aqueous solubility also complicates the manufacturing of a drug product and can lead to variability in the amount of drug substance in a formulation, or to reduced or inconsistent stability of the drug product on storage.

To combat these problems, poorly soluble drug substances are often formulated as suspensions. Accordingly, poorly soluble drug substances are generally sold as suspensions or as powders for reconstitution, which tend to be undesirable as they require a caregiver or patient to carry out a separate dispersion step. Even suspensions may require special handling by healthcare workers and caregivers. In addition, administration of large doses of the drug substance are needed to reach sufficient exposure to generate the desired therapeutic effect. However, administration of larger doses is associated with an increased risk of undesired side effects, including side effects attributable to variable exposure of the drug as well as high levels of solubilizing agents or co-solvents used in the formulation, which can cause irritation, allergic reactions, toxicity, or other safety risks.

Accordingly, there is a need in the art to develop new pharmaceutical compositions comprising CBD. The present disclosure aims to meet this need and/or provide other benefits, or at least provide the public with a useful choice. Certain embodiments included in this disclosure are pharmaceutical formulations comprising CBD that are free of organic solvents and include limited concentrations of excipients.

The following exemplary embodiments are provided. In one embodiment is an aqueous pharmaceutical composition comprising cannabidiol (CBD), a first surfactant, and a second surfactant, wherein the first surfactant and the second surfactant are different. In some aspects, the weight ratio of CBD to first surfactant in the composition is from about 1:5 to about 5:1. In some aspects, the weight ratio of the first surfactant to the second surfactant in the composition is from about 5:1 to about 1:5. In some aspects, the composition is from about 0.01 to about 15% CBD w/v (g/mL). In some aspects, the composition is a CBD concentrate, and comprises from about 4% to about 12% CBD w/v (g/mL). In some aspects, the composition is a CBD diluted concentrate, and comprises from about 0.01% to about 0.5% CBD w/v (g/mL). In an embodiment, the weight percentage of water in the composition is from about 50% to about 99.95%. In an embodiment, the weight percentage of water in a CBD concentrate composition is in the range of about 80% to about 95%. In an embodiment, the weight percentage of water in a CBD diluted concentrate composition is in the range of about 95% to 99.95%. In some embodiments, the pharmaceutical composition is free of organic solvents.

In another embodiment, the disclosure provides a method of making an aqueous CBD pharmaceutical composition comprising: combining CBD with a first surfactant, water, and an organic solvent to form a first solution; removing the organic solvent from the first solution to form a second solution; and adding a second surfactant to the second solution to obtain a CBD concentrate. In some aspects, the disclosure provides a method of making a CBD diluted concentrate comprising diluting a CBD concentrate with a diluent. In some aspects, the methods of making comprise filtering the CBD concentrate.

In an embodiment, an aqueous pharmaceutical composition, CBD concentrate, CBD diluted concentrate, first solution, or second solution is a solution. In some aspects, an aqueous pharmaceutical composition, CBD concentrate, CBD diluted concentrate, first solution, or second solution is a micellar solution. In some aspects, an aqueous pharmaceutical composition, CBD concentrate, CBD diluted concentrate, first solution, or second solution is a micellar suspension.

In another embodiment, the disclosure provides a method of treating a condition, disease, or disorder in a subject in need thereof, comprising administering to the subject a therapeutically effective amount of an aqueous pharmaceutical composition comprising CBD as described herein. In some aspects, the method comprises diluting a CBD concentrate to form a CBD diluted concentrate, and administering to the subject a therapeutically effective amount of the CBD diluted concentrate.

Furthermore, the following embodiments are specifically disclosed. Embodiment 1 is an aqueous composition comprising cannabidiol (CBD), a first surfactant, and a second surfactant, wherein the first surfactant and the second surfactant are different.

Embodiment 2 is the composition of embodiment 1, wherein the weight ratio of CBD to the first surfactant in the composition is from about 1:5 to about 5:1.

Embodiment 2a is the composition of embodiment 1, wherein the weight ratio of CBD to the first surfactant in the composition is from about 1:5 to about 1:4.

Embodiment 2b is the composition of embodiment 1, wherein the weight ratio of CBD to the first surfactant in the composition is from about 1:4 to about 1:3.

Embodiment 2c is the composition of embodiment 1, wherein the weight ratio of CBD to the first surfactant in the composition is from about 1:3 to about 1:2.

Embodiment 2d is the composition of embodiment 1, wherein the weight ratio of CBD to the first surfactant in the composition is from about 1:2 to about 1:1.

Embodiment 2e is the composition of embodiment 1, wherein the weight ratio of CBD to the first surfactant in the composition is from about 1:1 to about 2:1.

Embodiment 2f is the composition of embodiment 1, wherein the weight ratio of CBD to the first surfactant in the composition is from about 2:1 to about 3:1.

Embodiment 2g is the composition of embodiment 1, wherein the weight ratio of CBD to the first surfactant in the composition is from about 3:1 to about 4:1.

Embodiment 2h is the composition of embodiment 1, wherein the weight ratio of CBD to the first surfactant in the composition is from about 4:1 to about 5:1.

Embodiment 3 is the composition of any one of the preceding embodiments, wherein the weight ratio of the first surfactant to the second surfactant in the composition is from about 5:1 to about 1:5.

Embodiment 3a is the composition of embodiment 3, wherein the weight ratio of the first surfactant to the second surfactant in the composition is from about 1:5 to about 1:4.

Embodiment 3b is the composition of embodiment 3, wherein the weight ratio of the first surfactant to the second surfactant in the composition is from about 1:4 to about 1:3.

Embodiment 3c is the composition of embodiment 3, wherein the weight ratio of the first surfactant to the second surfactant in the composition is from about 1:3 to about 1:2.

Embodiment 3d is the composition of embodiment 3, wherein the weight ratio of the first surfactant to the second surfactant in the composition is from about 1:2 to about 1:1.

Embodiment 3e is the composition of embodiment 3, wherein the weight ratio of the first surfactant to the second surfactant in the composition is from about 1:1 to about 2:1.

Embodiment 3f is the composition of embodiment 3, wherein the weight ratio of the first surfactant to the second surfactant in the composition is from about 2:1 to about 3:1.

Embodiment 3g is the composition of embodiment 3, wherein the weight ratio of the first surfactant to the second surfactant in the composition is from about 3:1 to about 4:1.

Embodiment 3h is the composition of embodiment 3, wherein the weight ratio of the first surfactant to the second surfactant in the composition is from about 4:1 to about 5:1.

Embodiment 4 is the composition of any one of the preceding embodiments, wherein the weight ratio of CBD to the second surfactant in the composition is from about 1:5 to about 5:1.

Embodiment 4a is the composition of embodiment 4, wherein the weight ratio of CBD to the first surfactant in the composition is from about 1:5 to about 1:4.

Embodiment 4b is the composition of embodiment 4, wherein the weight ratio of CBD to the first surfactant in the composition is from about 1:4 to about 1:3.

Embodiment 4c is the composition of embodiment 4, wherein the weight ratio of CBD to the first surfactant in the composition is from about 1:3 to about 1:2.

Embodiment 4d is the composition of embodiment 4, wherein the weight ratio of CBD to the first surfactant in the composition is from about 1:2 to about 1:1.

Embodiment 4e is the composition of embodiment 4, wherein the weight ratio of CBD to the first surfactant in the composition is from about 1:1 to about 2:1.

Embodiment 4f is the composition of embodiment 4, wherein the weight ratio of CBD to the first surfactant in the composition is from about 2:1 to about 3:1.

Embodiment 4g is the composition of embodiment 4, wherein the weight ratio of CBD to the first surfactant in the composition is from about 3:1 to about 4:1.

Embodiment 4h is the composition of embodiment 4, wherein the weight ratio of CBD to the first surfactant in the composition is from about 4:1 to about 5:1.

Embodiment 5 is the composition of any one of the preceding embodiments, wherein the concentration of CBD in the composition is from about 0.01% to about 15% CBD w/v (g/mL).

Embodiment 5a is the composition of embodiment 5, wherein the concentration of CBD in the composition is from about 0.01% to about 0.1% CBD w/v (g/mL).

Embodiment 5b is the composition of embodiment 5, wherein the concentration of CBD in the composition is from about 0.1% to about 0.2% CBD w/v (g/mL).

Embodiment 5c is the composition of embodiment 5, wherein the concentration of CBD in the composition is from about 0.2% to about 0.5% CBD w/v (g/mL).

Embodiment 5d is the composition of embodiment 5, wherein the concentration of CBD in the composition is from about 0.5% to about 1% CBD w/v (g/mL).

Embodiment 5e is the composition of embodiment 5, wherein the concentration of CBD in the composition is from about 1% to about 2% CBD w/v (g/mL).

Embodiment 5f is the composition of embodiment 5, wherein the concentration of CBD in the composition is from about 2% to about 3% CBD w/v (g/mL).

Embodiment 5g is the composition of embodiment 5, wherein the concentration of CBD in the composition is from about 3% to about 4% CBD w/v (g/mL).

Embodiment 5h is the composition of embodiment 5, wherein the concentration of CBD in the composition is from about 4% to about 5% CBD w/v (g/mL).

Embodiment 5i is the composition of embodiment 5, wherein the concentration of CBD in the composition is from about 4% to about 5% CBD w/v (g/mL).

Embodiment 5j is the composition of embodiment 5, wherein the concentration of CBD in the composition is from about 5% to about 6% CBD w/v (g/mL).

Embodiment 5k is the composition of embodiment 5, wherein the concentration of CBD in the composition is from about 6% to about 7% CBD w/v (g/mL).

Embodiment 5l is the composition of embodiment 5, wherein the concentration of CBD in the composition is from about 7% to about 8% CBD w/v (g/mL).

Embodiment 5m is the composition of embodiment 5, wherein the concentration of CBD in the composition is from about 8% to about 9% CBD w/v (g/mL).

Embodiment 5n is the composition of embodiment 5, wherein the concentration of CBD in the composition is from about 9% to about 10% CBD w/v (g/mL).

Embodiment 5o is the composition of embodiment 5, wherein the concentration of CBD in the composition is from about 10% to about 11% CBD w/v (g/mL).

Embodiment 5p is the composition of embodiment 5, wherein the concentration of CBD in the composition is from about 11% to about 12% CBD w/v (g/mL).

Embodiment 5q is the composition of embodiment 5, wherein the concentration of CBD in the composition is from about 12% to about 13% CBD w/v (g/mL).

Embodiment 5r is the composition of embodiment 5, wherein the concentration of CBD in the composition is from about 13% to about 14% CBD w/v (g/mL).

Embodiment 5s is the composition of embodiment 5, wherein the concentration of CBD in the composition is from about 14% to about 15% CBD w/v (g/mL).

Embodiment 6 is the composition of embodiment 5, wherein the concentration of CBD in the composition is from about 0.01% to 0.5%, 0.5% to 1.5%, 1.5% to 2.5%, 2.5% to 3.5%, 3.5% to 4.5%, 4.5% to 5.5%, 5.5% to 6.5%, 6.5% to 7.5%, 7.5% to 8.5%, 8.5% to 9.5%, 9.5% to 10.5%, 10.5% to 11.5%, 11.5% to 12.5%, 12.5% to 13.5%, 13.5% to 14.5%, or 14.5% to 15%.

Embodiment 7 is the composition of any one of the preceding embodiments, wherein the first surfactant is a hydrophilic, non-ionic surfactant.

Embodiment 8 is the composition of embodiment 7, wherein the first surfactant is poloxamer 407 or a combination of surfactants comprising poloxamer 407, optionally wherein the combination further comprises poloxamer 188.

Embodiment 8a is the composition of embodiment 7, wherein the first surfactant consists of poloxamer 407.

Embodiment 8b is the composition of embodiment 7, wherein the first surfactant is a combination of surfactants comprising poloxamer 407.

Embodiment 8c is the composition of embodiment 7, wherein the first surfactant is a combination of surfactants comprising poloxamer 407 and poloxamer 188.

Embodiment 9 is the composition of embodiment 7, wherein the first surfactant is or comprises poloxamer 338.

Embodiment 10 is the composition of any one of the preceding embodiments, wherein the second surfactant is a hydrophilic, non-ionic surfactant.

Embodiment 11 is the composition of embodiment 10, wherein the second surfactant comprises a PEGylated castor oil, a PEGylated hydrogenated castor oil, a polyoxyethylene ester of a hydroxylated long-chain, saturated fatty acid, or a polyoxyl castor oil, or a combination thereof.

Embodiment 11a is the composition of any one of the preceding embodiments, wherein the second surfactant comprises a PEGylated castor oil.

Embodiment 11b is the composition of embodiment 11a, wherein the PEGylated castor oil is PEG 35 castor oil.

Embodiment 11c is the composition of any one of the preceding embodiments, wherein the second surfactant comprises a PEGylated hydrogenated castor oil.

Embodiment 11d is the composition of embodiment 11c, wherein the PEGylated hydrogenated castor oil is PEG 40 hydrogenated castor oil.

Embodiment 11e is the composition of any one of the preceding embodiments, wherein the second surfactant comprises a polyoxyethylene ester of a hydroxylated long-chain saturated fatty acid.

Embodiment 11f is the composition of embodiment 11e, wherein the polyoxyethylene ester of a hydroxylated long-chain saturated fatty acid is a poly-oxyethylene ester of 12-hydroxystearic acid.

Embodiment 11g is the composition of any one of the preceding embodiments, wherein the second surfactant comprises a polyoxyl castor oil.

Embodiment 11h is the composition of embodiment 11g, wherein the polyoxyl castor oil is polyoxyl 35 castor oil.

Embodiment 12 is the composition of embodiment 10 or 11, wherein the second surfactant comprises a polysorbate, optionally wherein the polysorbate is polysorbate 80.

Embodiment 13 is the composition of any one of embodiments 10-12, wherein the second surfactant comprises PEG 35 castor oil.

Embodiment 14 is the composition of any one of embodiments 10-13, wherein the second surfactant comprises Solutol HS-15.

Embodiment 15 is the composition of any one of embodiments 10-14, wherein the second surfactant comprises d-α-tocopheryl polyethylene glycol 1000 succinate.

Embodiment 16 is the composition of any one of embodiments 10-15, wherein the second surfactant comprises PEG 40 Hydrogenated Castor Oil.

Embodiment 17 is the composition of any one of the preceding embodiments, wherein the composition is an aqueous micellar solution.

Embodiment 18 is the composition of embodiment 17, wherein the Z-average particle size is less than about 50 nm, less than about 40 nm, less than about 30 nm, less than about 20 nm, or less than about 10 nm.

Embodiment 19 is the composition of any one of the preceding embodiments, which is free of organic solvents, and/or wherein the composition is a pharmaceutical composition.

Embodiment 20 is the composition of any one of the preceding embodiments, wherein the concentration of the first surfactant is 2-10 wt %.

Embodiment 21 is the composition of any one of the preceding embodiments, wherein the concentration of the second surfactant is 5-15 wt %.

Embodiment 22 is the composition of any one of the preceding embodiments, wherein the weight ratio of CBD to the first surfactant is in the range of 1:2 to 3:1.

Embodiment 23 is the composition of any one of the preceding embodiments, wherein the weight ratio of CBD to the second surfactant is in the range of 1:2 to 2:1.

Embodiment 24 is the composition of any one of the preceding embodiments, further comprising a buffer.

Embodiment 25 is the composition of embodiment 24, wherein the buffer is a citrate buffer or a phosphate buffer.

Embodiment 26 is the composition of any one of the preceding embodiments, having a pH in the range of 3-7, optionally wherein the pH is in the range of 3-6, 3-5, 3.5-4.5, or 3.75-4.25, or wherein the pH is about 4.

Embodiment 27 is a method of making the aqueous composition of any one of embodiments 1 to 25, comprising:
forming a first solution comprising CBD, a first surfactant, water, and an organic solvent;
removing the organic solvent from the first solution to form a second solution; and
adding a second surfactant to the second solution to obtain the aqueous composition.

Embodiment 28 is the method of embodiment 27, wherein removing the organic solvent comprises distillation.

Embodiment 29 is the method of embodiment 27 or 28, wherein removing the organic solvent comprises rotary evaporation.

Embodiment 30 is the method of any one of embodiments embodiment 27-29, wherein the organic solvent is or comprises ethanol, optionally wherein the ethanol is present in the first solution in a concentration of 15% to 60% by weight.

Embodiment 31 is the method of embodiment 30, wherein the organic solvent is or comprises one or more of an alcohol, alkane, ether, ester, or ketone.

Embodiment 32 is the method of embodiment 30, wherein the organic solvent is or comprises one or more of ethanol, isopropanol, pentane, ethyl ether, acetone, or ethyl acetate.

Embodiment 33 is a method of making the aqueous composition of any one of embodiments 1 to 32, comprising:
forming a first mixture comprising CBD and a first surfactant as solids;
melting the CBD and the first surfactant; and
combining the first mixture with a solution comprising a second surfactant to obtain the aqueous composition.

Embodiment 34 is the method of embodiment 33, wherein melting the CBD and the first surfactant comprises heating the CBD and the first surfactant to a temperature at or above 66° C., optionally wherein the temperature is in the range of 66° C.-100° C., 66° C.-90° C., 66° C.-80° C., 66° C.-75° C., or the temperature is about 70° C.

Embodiment 35 is the method of embodiment 33 or 34, further comprising cooling the first mixture after melting the CBD and the first surfactant, e.g., to room temperature or a temperature in the range of 18° C.-40° C.

Embodiment 36 is the method of embodiment 33 or 34, wherein the temperature of the first mixture is at or above 40° C., 45° C., 50° C., 55° C., 60° C., 66° C., 68° C., or 70° C. when combined with the solution comprising the second surfactant.

Embodiment 37 is the method of embodiment 36, wherein the temperature of the first mixture when combined with the solution comprising the second surfactant is in the range of 66° C.-100° C., 66° C.-90° C., 66° C.-80° C., 66° C.-75° C., or the temperature is about 70° C.

Embodiment 38 is the method of any one of embodiments 33-37, further comprising adding water to the first mixture before melting.

Embodiment 39 is the method of any one of embodiments 33-37, wherein water is not added to the first mixture before melting.

Embodiment 40 is the method of any one of embodiments 33-39, wherein organic solvent is not added to the first mixture before melting.

Embodiment 41 is the method of any one of embodiments 27-40, further comprising filtering the CBD concentrate.

Embodiment 42 is the method of any one of embodiments 27-41, comprising diluting the CBD concentrate with a diluent to form a CBD diluted concentrate.

Embodiment 43 is the method of any one of embodiments 27-42, wherein at least one of the aqueous compositions, the CBD diluted concentrate, the first solution, or the second solution is a solution.

Embodiment 44 is the method of embodiment 43, wherein at least one of the CBD concentrate, CBD diluted concentrate, first solution, or second solution is a micellar solution.

Embodiment 44a is the method of any one of embodiments 33-44, wherein removing the organic solvent from the first solution to form a second solution comprises rotary vaporation.

Embodiment 44b is the method of any one of embodiments 33-44a, wherein removing the organic solvent from the first solution to form a second solution comprises distillation.

Embodiment 45 is a method of treating a condition, disease, or disorder in a subject in need thereof, comprising administering to the subject a therapeutically effective amount of the composition of any one of embodiments 1 to 26.

Embodiment 46 is a method of treating a condition, disease, or disorder in a subject in need thereof, comprising diluting the composition of any one of embodiments 1 to 26 in a pharmaceutically acceptable diluent, thereby forming a diluted composition, and administering a therapeutically effective amount of the diluted composition to the subject.

Embodiment 47 is the composition of any one of embodiments 1 to 26, for use in treating a condition, disease, or disorder.

Embodiment 48 is the method of embodiment 45 or 46 or the composition for use of embodiment 46, wherein the disease, disorder, or symptom is pain (such as neuropathic pain or cancer-related pain), spasticity, anxiety, cognition, a movement disorder, epilepsy (such as childhood epilepsy, such as Lennox-Gastaut syndrome or Dravet syndrome), or dementia (such as Alzheimer's disease, Vascular Dementia, Dementia with Lewy bodies (DLB), Parkinson's disease, Frontotemporal dementia or Huntington's disease).

Embodiment 49 is the method or composition for use of any one of embodiments 45-48, wherein the composition is a CBD diluted concentrate.

Embodiment 50 is a composition comprising water, a surfactant, an organic solvent, and CBD, wherein the CBD is dissolved in the composition.

Embodiment 51 is the composition of embodiment 50, wherein the surfactant is a hydrophilic, non-ionic surfactant.

Embodiment 52 is the composition of embodiment 50 or 51, wherein the surfactant comprises one or more of poloxamer 407 and poloxamer 188.

Embodiment 53 is the composition of any one of embodiments 50-52, wherein the surfactant comprises poloxamer 407.

Embodiment 54 is the composition of any one of embodiments 50-53, wherein the surfactant comprises poloxamer 188.

Embodiment 55 is the composition of any one of embodiments 50-54, wherein the organic solvent is ethanol.

Embodiment 56 is the composition of any one of embodiments 50-55, wherein the organic solvent is butane.

Embodiment 57 is the composition of any one of embodiments 50-56, wherein the organic solvent comprises one or more of an alcohol, alkane (such as a C₃, C₄, C₅, or C₆ alkane, such as butane or pentane), ether, ester, ketone, or any combination thereof, optionally wherein the organic solvent comprises ethanol, isopropanol, pentane, ethyl ether, acetone, ethyl acetate, or any combination thereof.

Embodiment 58 is the composition of any one of embodiments 50-57, wherein the organic solvent is present in a concentration of 30-40 wt %, 40-50 wt %, 50-60 wt %, or 60-70 wt %.

Embodiment 59 is the composition of any one of embodiments 50-58, wherein the concentration of CBD in the composition is 5-15 wt %, such as 5-6 wt %, 6-7 wt %, 7-8 wt %, 8-9 wt %, 9-10 wt %, 10-11 wt %, 11-12 wt %, 12-13 wt %, 13-14 wt %, or 14-15 wt %.

Embodiment 60 is the composition of any one of embodiments 50-59, wherein the concentration of the surfactant in the composition is 4-10 wt %, such as 4-5 wt %, 5-6 wt %, 6-7 wt %, 7-8 wt %, 8-9 wt %, or 9-10 wt %.

Embodiment 61 is a method of preparing a solvent-free composition comprising CBD, the method comprising removing the organic solvent from the composition of any one of embodiments 50-60.

Embodiment 62 is the method of embodiment 61, wherein removing the organic solvent comprises removal by distillation.

### II. BRIEF DESCRIPTION OF THE DRAWINGS

FIG. 1 shows photographs of compositions produced as described in Example 11. At left is the composition prepared by the disclosed process comprising distillation; at right is the comparative composition prepared by simple mixing, without using ethanol or distillation. Visible crystals of undissolved CBD are present in the comparative composition.

FIG. 2 shows the stability of CBD in a formulation according to the disclosure after three months at the indicated temperatures.

FIGS. 3A-B show the pharmacokinetics of CBD administered to rats i.v. or orally, respectively, using a formulation according to the disclosure.

### III. DETAILED DESCRIPTION

Reference will now be made in detail to certain embodiments of the invention. While the invention will be described in conjunction with the described embodiments, it will be understood that such descriptions are not intended to limit the invention to those embodiments. On the contrary, the invention is intended to cover all alternatives, modifications, and equivalents, which may be included within the invention as defined by the appended claims.

Before describing the present teachings in detail, it is to be understood that the disclosure is not limited to specific compositions or process steps, as such may vary. It should be noted that, as used in this specification and the appended claims, the singular form “a,” “an,” and “the” include plural references unless the context clearly dictates otherwise. Thus, for example, reference to “a surfactant” includes a plurality of surfactants and the like.

Numeric ranges are inclusive of the numbers defining the range. Measured and measurable values are understood to be approximate, taking into account significant digits and the error associated with the measurement. Also, the use of “comprise,” “comprises,” “comprising,” “contain,” “contains,” “containing,” “include,” “includes,” “included,” and “including” are not intended to be limiting. It is to be understood that both the foregoing general description and detailed description are exemplary and explanatory only and are not restrictive of the teachings.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The section headings used herein are for organizational purposes only and are not to be construed as limiting the desired subject matter in any way. In the event that any literature incorporated by reference contradicts any term defined in this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

A. Definitions

Unless otherwise defined herein, scientific and technical terms used herein have the meanings that are commonly understood by those of ordinary skill in the art. In the event of any latent ambiguity, definitions provided herein take precedence over any dictionary or extrinsic definition. Unless otherwise required by context, singular terms shall include pluralities and plural terms shall include the singular.

The terms “or a combination thereof” and “or combinations thereof” as used herein refers to any and all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

“Or” is used in the inclusive sense, i.e., equivalent to “and/or,” unless the context requires otherwise.

The term “localized” refers generally to dispersion of a poorly soluble drug in lipid vesicles, e.g., micelles.

The term “micellar solution” refers to a clear dispersion of micelles in aqueous solution.

The terms “micellar suspension” and “microemulsion” are used interchangeably and refer to a suspension of micelles in water. Microemulsions may be identified based on dynamic light scattering (DLS) analysis using methods known in the art.

The term “polydispersity index” or “PDI” means a number calculated from a simple 2 parameter fit to correlation data (the cumulants analysis of the DLS-measured intensity autocorrelation function). The Polydispersity Index is dimensionless and scaled such that values smaller than 0.05 are rarely seen other than with highly monodisperse standards. Values greater than 0.7 indicate that the sample has a very broad size distribution and is probably not suitable for the dynamic light scattering (DLS) technique. The various size distribution algorithms work with data that falls between these two extremes. The calculations for these parameters are defined in the ISO standard document 13321:1996 E and ISO 22412:2008. In the Cumulants analysis, a single particle size mode is assumed and a single exponential fit is applied to the autocorrelation function and the polydispersity describes the width of the assumed Gaussian distribution. In terms of a protein analysis, a % polydispersity less than 20% indicates that the sample is monodisperse.

The terms “solvent-free,” “free of organic solvents,” and “organic solvent-free” are used interchangeably to refer to compositions that do not include organic solvents or include less than a trace level of an organic solvent, such as 0.5%, 0.2%, or 0.1%. In some embodiments, a solvent-free composition may contain a trace amount of ethanol only, with no other organic solvents present at all. A composition initially containing an organic solvent susceptible to removal by distillation (e.g., ethanol) may be rendered solvent-free by undergoing distillation at a sufficient temperature to remove the organic solvent, e.g., 60° C. for ethanol under vacuum. Typically, it is sufficient to remove a mass of liquid slightly greater than the mass of organic solvent originally added to produce a solvent-free composition. For example, if 15 g of ethanol was originally present, removing 16 g of distillate generally suffices to render the composition solvent-free as defined herein.

The term “surfactant” means a compound that lowers the surface tension (or interfacial tension) between two liquids or between a liquid and a solid. Surfactants may act as detergents, wetting agents, emulsifiers, foaming agents, solubilizers, and/or dispersants.

The term “suspension” refers to a cloudy heterogeneous mixture (e.g., of micelles or other solids suspended in aqueous solution) that may settle over time.

The term “therapeutically effective amount” means an amount sufficient to achieve a clinically desired outcome, or to treat, relieve, or ameliorate a condition, disease, or disorder in a subject suffering from said condition, disease, or disorder.

B. Exemplary Aqueous Compositions

Provided herein are aqueous preparations of CBD and methods of preparing these compositions.

Provided herein are aqueous CBD compositions that include CBD, water, a first surfactant, and a second surfactant, wherein the first surfactant and the second surfactant are different. In some aspects, the weight ratio of CBD to first surfactant in the composition is from about 1:5 to about 5:1. In some aspects, the weight ratio of CBD to first surfactant is from about 3:1 to about 1:1. In some aspects, the weight ratio of CBD to first surfactant is from about 2:1 to about 1:1. In some aspects, the weight ratio of CBD to first surfactant is about 1:1, or about 1.1:1, or about 1.2:1, or about 1.3:1, or about 1.33:1, or about 1.375:1, or about 1.5:1, or about 1.57:1, or about 1.6:1. In some aspects, the weight ratio of CBD to first surfactant is from about 1:1 to about 1:5. In some aspects, the weight ratio of CBD to first surfactant is from about 1:1 to about 1:2. In some aspects, the weight ratio of CBD to first surfactant is about 1:1.25. In some embodiments, the weight ratio of CBD to first surfactant is in the range of 1:2 to 3:1. In some embodiments, the weight ratio of CBD to first surfactant is in the range of 1:2 to 1.5:2, 1.5:2 to 1.75:2, 1.75:2 to 2:2 (or 1:1), 2:2 (or 1:1) to 2.25:2, 2.25:2 to 2.5:2, 2.5:2 to 2.75:2, 2.75:2 to 3:2, 3:2 to 3.25:2, 3.25:2 to 3.5:2, 3.5:2 to 3.75:2, 3.75:2 to 4:2 (or 2:1), 4:2 (or 2:1) to 4.25:2, 4.25:2 to 4.5:2, 4.5:2 to 4.75:2, 4.75:2 to 5:2, 5:2 to 5.25:2, 5.25:2 to 5.5:2, 5.5:2 to 5.75:2, or 5.75:2 to 6:2 (or 3:1). In some embodiments, the weight ratio of CBD to second surfactant is in the range of 1:2 to 2:1. In some embodiments, the weight ratio of CBD to second surfactant is in the range of 1:2 to 1.5:2, 1.5:2 to 1.75:2, 1.75:2 to 2:2 (or 1:1), 2:2 (or 1:1) to 2.25:2, 2.25:2 to 2.5:2, 2.5:2 to 2.75:2, 2.75:2 to 3:2, 3:2 to 3.25:2, 3.25:2 to 3.5:2, 3.5:2 to 3.75:2, or 3.75:2 to 4:2 (or 2:1).

In some aspects, the weight ratio of first surfactant to second surfactant is from about 1:1 to about 1:5. In some aspects, the weight ratio of first surfactant to second surfactant is from about 1:1 to about 1:3. In some aspects, the weight ratio of first surfactant to second surfactant is from about 1:1.5 to about 1:3. In some aspects, the weight ratio of first surfactant to second surfactant is from about 1:1.5 to about 1:2. In some aspects, the weight ratio of first surfactant to second surfactant is about 1:1.8. In some aspects, the weight ratio of first surfactant to second surfactant is from about 1:2 to about 1:3. In some aspects, the weight ratio of first surfactant to second surfactant is about 1:2, or about 1:2.2, or about 1:2.25, or about 1:2.4, or about 1:2.57, or about 1:2.6. In some aspects, the weight ratio of first surfactant to second surfactant is from about 5:1 to about 1:1. In some aspects, the weight ratio of first surfactant to second surfactant is from about 2:1 to about 1:1. In some aspects, the weight ratio of first surfactant to second surfactant is about 1.5:1.

In an embodiment, the weight percentage (g/mL) of CBD in the composition is in the range of about 0.01% to about 15%. In certain embodiments, the weight percentage of CBD is in the range of 0.1 to 1%, 1 to 10%, or 10-15%. In some aspects, the composition is a CBD concentrate, and comprises from about 4% to about 12% CBD by weight, or about 4% to about 10%, or about 4%, or about 5%, or about 6%, or about 7%, or about 8%, or about 9%, or about 10%. In some aspects, a CBD concentrate comprises 5.0%, or 5.7%, or 5.8%, or 6.0%, or 6.1%, or 6.2%, or 6.3%, or 6.4%, or 6.6% CBD by weight. In some aspects, a CBD concentrate comprises 10% CBD by weight. In some aspects, the composition is a CBD diluted concentrate, and comprises from about 0.01% to about 0.5% CBD by weight. In some aspects, a CBD diluted concentrate comprises from about 0.01% to about 0.15% CBD by weight. In some aspects, a CBD diluted concentrate comprises 0.015% CBD by weight, or comprises 0.1% CBD by weight.

In an embodiment, the weight percentage of CBD in the composition is in the range of 0.1 to 20%. In certain embodiments, the weight percentage of CBD is in the range of 0.1 to 1%, 1 to 10%, 10 to 15%, or 15 to 20%. In some embodiments, the weight percentage of CBD is from about 0.01% to 0.5%, 0.5% to 1.5%, 1.5% to 2.5%, 2.5% to 3.5%, 3.5% to 4.5%, 4.5% to 5.5%, 5.5% to 6.5%, 6.5% to 7.5%, 7.5% to 8.5%, 8.5% to 9.5%, 9.5% to 10.5%, 10.5% to 11.5%, 11.5% to 12.5%, 12.5% to 13.5%, 13.5% to 14.5%, or 14.5% to 15%. Expressed in weight/volume terms, the concentration of CBD in an aqueous composition is in the range of 0.05 to 300 mg/mL. For a CBD concentrate, the CBD concentration is in the range of 10 to 100 mg/mL, or 25 to 75 mg/mL, or 40 to 75 mg/mL. For a diluted CBD concentrate, the CBD concentration is in the range of 0.05 to 5 mg/mL, or 0.1 to 2 mg/mL, or 0.05 to 3 mg/mL.

In an embodiment, the weight percentage of water in the composition is from about 50% to about 99.95%. In an embodiment, the weight percentage of water in a CBD concentrate composition is in the range of about 80% to about 95%. In certain embodiments, the weight percentage of water is in the range of 50 to 60%, 60 to 70%, 70 to 80%, 80 to 90%, or 90 to 99.5%. In an embodiment, the weight percentage of water in a CBD diluted concentrate composition is in the range of about 95% to 99.95%. In some aspects, the weight percentage of water is 100% minus the weight percentage of other ingredients in the formulation.

In an embodiment, the composition is a CBD concentrate, and the weight percentage (w/w) of the first surfactant in the composition is in the range of 0.02 to 30%. In another embodiment, the weight percentage of the first surfactant is in the range of 0.25% to 10%. In some aspects, the weight percentage of the first surfactant is from about 1% to about 10%. In some aspects, the weight percentage of the first surfactant is from about 4% to about 10%. In some aspects, the weight percentage of the first surfactant is from about 4% to about 6%. In some aspects, the weight percentage of the first surfactant is about 4%, about 5%, about 6%, about 7%, about 8%, or about 9%. In some aspects, the weight percentage of the first surfactant is from about 5% to about 6%. In some aspects, the weight percentage of the first surfactant is about 3.9%, about 4.5%, about 5.0%, about 5.5%, about 5.6%, or about 9.0%.

In some aspects, the composition is a CBD diluted concentrate, and comprises the above weight percentages for the first surfactant, diluted by a factor of up to 500.

In an embodiment, the first surfactant is a hydrophilic, non-ionic surfactant. In some embodiments, the first surfactant is a poloxamer. Additional examples of hydrophilic surfactants include those comprising ethoxylated glyceryl ester functionality, polyethylene glycol units, polypropylene glycol units, alkylglycoside functionality, etc., or combinations thereof. Non-ionic surfactants include those lacking amine, sulfate, phosphate, phosphonate, and carboxylate functionalities, or more generally functionalities that are substantially anionic or cationic at physiological pH such as pH 7.4 in water or phosphate-buffered saline. Hydrophilic surfactants may comprise a hydrocarbon component (e.g., an optionally substituted C1-C18 aliphatic chain) provided that they also comprise sufficient electronegative or electropositive moieties (e.g., alcohols, amides, carbonyls, and other hydrogen bond donors/acceptors) to confer substantial solubility in water.

In some aspects, the first surfactant is an ethoxylated glyceryl ester. In some aspects, the first surfactant is a copolymer comprising polyethylene glycol units. In some aspects, the first surfactant is a copolymer of polyethylene glycol and polypropylene glycol. In some aspects, the first surfactant is a poloxamer. A poloxamer is a non-ionic surfactant that is a tri-block copolymer with a central polypropylene glycol portion and polyethylene glycol termini. In some aspects, the first surfactant is poloxamer 407. In some aspects, the first surfactant is a combination of surfactants comprising poloxamer 407 and an additional surfactant. In some aspects, the first surfactant is poloxamer 188. In some aspects, the first surfactant is a combination of surfactants comprising poloxamer 188 and an additional surfactant. In some aspects, the first surfactant is poloxamer 407 and poloxamer 188. In some aspects, the first surfactant is poloxamer 407. In some aspects, the first surfactant is a combination of surfactants comprising poloxamer 407 and an additional surfactant.

In some embodiments, the concentration of the first surfactant is 2-10 wt %. In some embodiments, the concentration of the first surfactant is 5-15 wt %. In some embodiments, the concentration of the first surfactant is about 2-2.5 wt %, 2.5-3 wt %, 3-3.5 wt %, 3.5-4 wt %, 4-4.5 wt %, 4.5-5 wt %, 5-5.5 wt %, 5.5-6 wt %, 6-6.5 wt %, 6.5-7 wt %, 7-7.5 wt %, 7.5-8 wt %, 8-8.5 wt %, 8.5-9 wt %, 9-9.5 wt %, 9.5-10 wt %, 10-10.5 wt %, 10.5-11 wt %, 11-11.5 wt %, 11.5-12 wt %, 12-12.5 wt %, 12.5-13 wt %, 13-13.5 wt %, 13.5-14 wt %, 14-14.5 wt %, or 14.5-15 wt %.

In an embodiment, the composition is a CBD concentrate, and the weight percentage (w/w) of the second surfactant in the composition is from about 1% to about 30%. In some aspects, the weight percentage of the second surfactant is from about 5% to about 15%. In some aspects, the weight percentage of the second surfactant is from about 10% to about 15%. In some aspects, the weight percentage of the second surfactant is about 6%, or is about 10%, or is about 12.5%, or is about 13%. In some aspects, the weight percentage of the second surfactant is about 10%. In some aspects, the composition is a CBD diluted concentrate, and comprises the above weight percentages of the second surfactant, diluted by a factor of up to 500.

In an embodiment, the second surfactant is a hydrophilic, non-ionic surfactant. In some aspects, the second surfactant is a PEGylated castor oil, a PEGylated hydrogenated castor oil, a polyoxyethylene ester of a hydroxylated long-chain, saturated fatty acid, or a polyoxyl castor oil, or a combination thereof. In some aspects, the second surfactant is or comprises one or more of PEG 40 hydrogenated castor oil, PEG 30 hydrogenated castor oil, polyethylene glycol (15)-hydroxystearate (Solutol HS-15), PEG 35 castor oil, PEG 30 castor oil, PEG 33 castor oil, PEG 36 castor oil, PEG 40 castor oil, polyoxyl 35 castor oil, polyoxyl 30 castor oil, polyoxyl 40 castor oil, polyoxyl 40 hydrogenated castor oil, polyoxyl 60 hydrogenated castor oil, a polysorbate such as polysorbate 20 or polysorbate 80, d-α-tocopheryl polyethylene glycol 1000 succinate (TPGS), PEG 300 caprylic/capric glycerides (SOFTIGEN 767), PEG 400 caprylic/capric glycerides (LABRASOL), PEG 300 oleic glycerides (LABRAFIL M-1944CS), PEG 300 linoleic glycerides (LABRAFIL M-2125CS), polyoxyl 8 stearate (PEG 400 monosterate), or polyoxyl 40 stearate (PEG 1750 monosterate). In some embodiments, the second surfactant is PEG 40 hydrogenated castor oil, polyethylene glycol (15)-hydroxystearate, PEG 35 castor oil, or polyoxyl 35 castor oil, or a combination thereof. In some aspects, the second surfactant is PEG 40 hydrogenated castor oil. In some aspects, the second surfactant is polyethylene glycol (15)-hydroxystearate. In some aspects, the second surfactant is PEG 35 castor oil. In some aspects, the second surfactant is PEG 40 hydrogenated castor oil and polyoxyl 35 castor oil. In some aspects, the second surfactant is polyethylene glycol (15)-hydroxystearate and polyoxyl 35 castor oil. In some aspects, the second surfactant is a polysorbate. In some aspects, the second surfactant is polysorbate 80. In some aspects, the second surfactant is Solutol HS-15. In some aspects, the second surfactant is d-α-tocopheryl polyethylene glycol 1000 succinate (TPGS).

In an embodiment, the weight ratio of the first surfactant to the second surfactant is in the range of about 0.1 to 50. In another embodiment, the weight ratio of the first surfactant to the second surfactant is in the range of about 0.01 to about 20. In another embodiment, the weight ratio of the first surfactant to the second surfactant is from about 1:5 to about 5:1. In another embodiment, the weight ratio of the first surfactant to the second surfactant is in the range of about 3:1 to 1:1. In some aspects, the weight ratio of the first surfactant to the second surfactant is about 1.5:1. In other aspects, the weight ratio of the first surfactant to the second surfactant in from about 1:1 to about 1:3. In some aspects, the ratio is from about 1:2 to about 1:3. In some aspects, the ratio is about 1:1.8, or about 1:2, or about 1:2.2, or about 1:2.4.

In some embodiments, representative CBD aqueous formulations comprise: CBD (about 0.01% to about 15% by weight (w/v)), a first surfactant (about 1% to about 10% by weight (w/w)), a second surfactant (about 1% to about 30% by weight (w/w)), and water. In some embodiments, CBD aqueous formulations comprise: CBD (about 4% to about 12% by weight (w/v)), a first surfactant (about 4% to about 10% by weight (w/w)), a second surfactant (about 5% to about 15% by weight (w/w)), and water. In some embodiments, a CBD aqueous composition comprises CBD (up to 10% by weight (w/v)), a first surfactant that is one or more poloxamers (0.1 to 5% by weight (w/w)), and a second surfactant that is an ethoxylated glyceryl ester, PEG 40 hydrogenated castor oil, PEG 35 castor oil, or poly-oxyethylene ester of 12-hydroxystearic acid, or a combination thereof (1 to 30% by weight (w/w)).

In an embodiment, the composition is homogeneous at temperatures in the range of 0 to 30° C. In certain embodiments, the composition is homogeneous at temperatures in the range of 0 to 10° C., or 10 to 20° C., or 20 to 30° C. In a particular embodiment, the composition is homogeneous at 25° C.

In an embodiment, the composition is a micellar composition at temperatures in the range of 0 to 30° C. In certain embodiments, the composition is a micellar composition at temperatures in the range of 0 to 10° C., or 10 to 20° C., or 20 to 30° C. In a particular embodiment, the composition is a micellar composition at 25° C.

In an embodiment, the composition is an aqueous micellar solution, wherein CBD is localized in the micelles. In an embodiment, the composition is an aqueous micellar solution, wherein CBD is localized in the micelles at temperatures in the range of 0 to 30° C. In certain embodiments, the composition is an aqueous micellar solution, wherein CBD is localized in the micelles at temperatures in the range of 0 to 10° C., or 10 to 20° C., or 20 to 30° C. In a particular embodiment, the composition is an aqueous micellar solution, wherein CBD is localized in the micelles, at 25° C.

In an embodiment, the Z-average (d·nm) of the micelles is about 5 to 500 nm, 10 to 200 nm, 5 to 200 nm, 10 to 200 nm, 5 to 100 nm, 5 to 50 nm, about 5 to 40 nm, about 5 to 30 nm, about 5 to 20 nm, 10 to 100 nm, 10 to 50 nm, about 10 to 40 nm, about 10 to 30 nm, or about 10 to 20 nm. In an embodiment, the Z-average (d·nm) of the micelles is about 15 to 500 nm, 15 to 200 nm, 15 to 100 nm, 15 to 50 nm, about 15 to 40 nm, or about 15 to 30 nm. In an embodiment, the Z-average (d·nm) of the micelles is about 20 to 500 nm, 20 to 200 nm, 20 to 100 nm, 20 to 50 nm, about 20 to 40 nm, or about 20 to 30 nm. In some embodiments, the Z-average particle size of the micelles in solution is less than about 50 nm, less than about 40 nm, less than about 30 nm, less than about 20 nm, or less than about 10 nm. The Z-average is the intensity weighted mean hydrodynamic size of the ensemble collection of particles measured by dynamic light scattering (DLS). The Z-average is derived from a Cumulants analysis of the measured correlation curve, wherein a single particle size is assumed, and a single exponential fit is applied to the autocorrelation function. In an embodiment, the associated temperature (i.e., the temperature of the micellar solution) is in the range of 0 to 30° C. In certain embodiments, the associated temperature is in the range of 0 to 10° C., or 10 to 20° C., or 20 to 30° C. In a particular embodiment, the associated temperature is 25° C.

In an embodiment, the compositions described herein further comprise one or more excipients. Non-limiting examples of excipients include propylene glycol, glycerin, ethanol, polyethylene glycol (300 and 400), sorbitol, and dimethylacetamide. Non-limiting examples of additives and excipients include dimethyl sulfoxide (DMSO), tocopherols and derivatives thereof, tocotrienols and derivatives thereof, polysorbates, hyaluronic acid and derivatives thereof, cyclodextrins, mannitol, sorbitol, sodium chloride, EDTA, monobasic sodium phosphate monohydrate, dibasic sodium phosphate heptahydrate, dextrose, and sucrose. Exemplary excipients also include diluents, for example, for dilution of a CBD concentrate to a CBD diluted concentrate. Suitable diluents include water, buffered saline (e.g., Dulbecco's Phosphate Buffered Saline (DPBS)), carbonated beverages such as carbonated water or carbonated soda, tea beverages, or fruit juice. Carbonated or tea beverages may include natural and/or artificial sweeteners and flavorings.

The aqueous composition may have any pH suitable for administration to a subject. In some embodiments, the aqueous CBD composition has a pH in the range of 4 to 9, such as 5 to 9, 4 to 8, 5 to 8, 5 to 7, or 6 to 8. In some embodiments, the aqueous CBD composition has a pH in the range of 6 to 7.6. In some embodiments, the pH is in the range of 6 to 6.4, 6.3 to 6.7, 6.4 to 6.8, 6.8 to 7.2, 7 to 7.4, or 7.2 to 7.6. In some embodiments, the pH is from 6.5 to 7.2. In some embodiments, the pH is in the range of 3-7. In some embodiments, the pH is in the range of 3-6, 3-5, 3.5-4.5, or 3.75-4.25. In some embodiments, the pH is about 4. The aqueous composition may comprise a buffer, e.g., a phosphate or citrate buffer. The buffer may be pharmaceutically acceptable. In some embodiments, the buffer is present at a concentration in the range of 5-100 mM, such as 10-50 mM, 15-30 mM, or about 20 mM.

In some embodiments, the composition is stable for at least 25 days, 30 days, 6 months, 1 year, or 2 years. In some embodiments, the pharmaceutical composition is stable at room temperature for at least 25 days, 30 days, 6 months, 1 year, or 2 years. In some embodiments, the pharmaceutical composition is stable at 2-8° C. for at least 25 days, 30 days, 6 months, 1 year, or 2 years. In some embodiments, the pharmaceutical composition is stable at 37-42° C. for at least 25 days, 30 days, 6 months, 1 year, or 2 years.

In some embodiments, the aqueous composition is a pharmaceutical composition (e.g., is sterile, and/or comprises water for injection or another pharmaceutically acceptable carrier). In some embodiments, aqueous CBD compositions may be formulated in a suitable unit dosage form for administration to a subject. In such form, the preparation is subdivided into unit doses containing appropriate quantities of the active component. The unit dosage form can be a packaged preparation, the package containing discrete quantities of formulation. For example, the aqueous compositions may be loaded into capsules, syringes, ampules, depot devices, aerosol delivery devices, or other drug delivery devices.

Further details on techniques for formulation and administration may be found in Gennaro, A., Ed., Remington's Pharmaceutical Sciences, 18th Ed. (1990) (Mack Publishing Co., Easton, Pa.).

### Exemplary Embodiments

In a particular embodiment of the composition described herein, the first surfactant is Poloxamer 407 and the second surfactant is PEG 40 hydrogenated castor oil. See, e.g., Examples 1 and 3. In some embodiments, Poloxamer 407 is present in any composition described herein in a concentration described in Example 1 or Example 3, or in about a concentration described in Example 1 or Example 3. In some embodiments, PEG 40 hydrogenated castor oil is present in any composition described herein in a concentration described in Example 1 or Example 3, or in about a concentration described in Example 1 or Example 3. In some embodiments, Poloxamer 407 and PEG 40 hydrogenated castor oil are present in any composition described herein in concentrations described for Poloxamer 407 and PEG 40 hydrogenated castor oil, respectively, in Example 1 or Example 3, or in about concentrations described in Example 1 or Example 3.

In a particular embodiment, the first surfactant is Poloxamer 407 and the second surfactant is Solutol HS-15. See, e.g., Examples 2, 9, and 10. In some embodiments, Poloxamer 407 is present in any composition described herein in a concentration described in Example 2, 9, or 10, or in about a concentration described in Example 2, 9, or 10. In some embodiments, Solutol HS-15 is present in any composition described herein in a concentration described in Example 2, 9, or 10, or in about a concentration described in Example 2, 9, or 10. In some embodiments, Poloxamer 407 and Solutol HS-15 are present in any composition described herein in concentrations described for Poloxamer 407 and Solutol HS-15, respectively, in Example 2, 9, or 10, or in about concentrations described in Example 2, 9, or 10.

In a particular embodiment, the first surfactant is Poloxamer 407 and the second surfactant is PEG 35 castor oil. See, e.g., Example 4. In some embodiments, Poloxamer 407 is present in any composition described herein in a concentration described in Example 4, or in about a concentration described in Example 4. In some embodiments, PEG 35 castor oil is present in any composition described herein in a concentration described in Example 4., or in about a concentration described in Example 4 In some embodiments, Poloxamer 407 and PEG 35 castor oil are present in any composition described herein in concentrations described for Poloxamer 407 and PEG 35 castor oil, respectively, in Example 4, or in about concentrations described in Example 4.

In a particular embodiment, the first surfactant is Poloxamer 407 and the second surfactant is Polyoxyl 35 castor oil and PEG 40 hydrogenated castor oil. See, e.g., Example 5. In some embodiments, Poloxamer 407 is present in any composition described herein in a concentration described in Example 5, or in about a concentration described in Example 5. In some embodiments, Polyoxyl 35 castor oil is present in any composition described herein in a concentration described in Example 5, or in about a concentration described in Example 5. In some embodiments, PEG 40 hydrogenated castor oil is present in a concentration described in Example 5, or in about a concentration described in Example 5. In some embodiments, Poloxamer 407, Polyoxyl 35 castor oil, and PEG 40 hydrogenated castor oil are present in any composition described herein in concentrations described for Poloxamer 407, Polyoxyl 35 castor oil, or PEG 40 hydrogenated castor oil, respectively, in Example 5, or in about concentrations described in Example 5.

In a particular embodiment, the first surfactant is Poloxamer 407 and the second surfactant is Polyoxyl 35 castor oil and Solutol HS-15. See, e.g., Examples 6 and 7. In some embodiments, Poloxamer 407 is present in any composition described herein in a concentration described in Example 6 or Example 7, or in about a concentration described in Example 6 or Example 7. In some embodiments, Polyoxyl 35 castor oil is present in any composition described herein in a concentration described in Example 6 or Example 7, or in about a concentration described in Example 6 or Example 7. In some embodiments, Solutol HS-15 is present in a concentration described in Example 6 or Example 7, or in about a concentration described in Example 6 or Example 7. In some embodiments, Poloxamer 407, Polyoxyl 35 castor oil, and Solutol HS-15 are present in any composition described herein in concentrations described for Poloxamer 407, Polyoxyl 35 castor oil, or Solutol HS-15, respectively, in Example 6 or Example 7, or in about concentrations described in Example 6 or Example 7.

In a particular embodiment, the first surfactant is Poloxamer 407 and Poloxamer 188, and the second surfactant is Polyoxyl 35 castor oil and Solutol HS-15. See, e.g., Example 8. In some embodiments, Poloxamer 407 is present in any composition described herein in a concentration described in Example 8, or in about a concentration described in Example 8. In some embodiments, Poloxamer 188 is present in any composition described herein in a concentration described in Example 8, or in about a concentration described in Example 8. In some embodiments, Polyoxyl 35 castor oil is present in any composition described herein in a concentration described in Example 8, or in about a concentration described in Example 8. In some embodiments, Solutol HS-15 is present in any composition described herein in a concentration described in Example 8, or in about a concentration described in Example 8. In some embodiments, Poloxamer 407, Poloxamer 188, Polyoxyl 35 castor oil, and Solutol HS-15 are present in any composition described herein in concentrations described for Poloxamer 407, Poloxamer 188, Polyoxyl 35 castor oil, and Solutol HS-15, respectively, in Example 8, or in about concentrations described in Example 8.

In a particular embodiment, the first surfactant is Poloxamer 407, and the second surfactant is Polysorbate 80. See, e.g., Examples 11, 16, 17, 18, 19, 20, 21, 23, 24, 25, 26, 32, 40, and 41. In some embodiments, the first surfactant is present in any composition described herein in a concentration described in Example 11, 16, 17, 18, 19, 20, 21, 23, 24, 25, 26, 32, 40, or 41, or in about a concentration described in Example 11, 16, 17, 18, 19, 20, 21, 23, 24, 25, 26, 32, 40, or 41. In some embodiments, the second surfactant is present in any composition described herein in a concentration described in Example 11, 16, 17, 18, 19, 20, 21, 23, 24, 25, 26, 32, 40, or 41, or in about a concentration described in Example 11, 16, 17, 18, 19, 20, 21, 23, 24, 25, 26, 32, 40, or 41. In some embodiments, the first and second surfactants are present in any composition described herein in concentrations described in Example 11, 16, 17, 18, 19, 20, 21, 23, 24, 25, 26, 32, 40, or 41, or in about concentrations described in Example 11, 16, 17, 18, 19, 20, 21, 23, 24, 25, 26, 32, 40, or 41. The first and/or second surfactants in any such embodiments may be those described in Example 11, 16, 17, 18, 19, 20, 21, 23, 24, 25, 26, 32, 40, or 41.

In a particular embodiment, the first surfactant is Poloxamer 407, and the second surfactant is Vitamin E TPGS. See, e.g., Example 12 and 33. In some embodiments, the first surfactant is present in any composition described herein in a concentration described in Example 12 or 33, or in about a concentration described in Example 12 or 33. In some embodiments, the second surfactant is present in any composition described herein in a concentration described in Example 12 or 33, or in about a concentration described in Example 12 or 33. In some embodiments, the first and second surfactants are present in any composition described herein in concentrations described in Example 12 or 33, or in about concentrations described in Example 12 or 33. The first and/or second surfactants in any such embodiments may be those described in Example 12 or 33.

In a particular embodiment, the first surfactant is Poloxamer 407, and the second surfactant is Polysorbate 80 and Vitamin E TPGS. See, e.g., Example 13. In some embodiments, the first surfactant is present in any composition described herein in a concentration described in Example 13, or in about a concentration described in Example 13. In some embodiments, the second surfactant is present in any composition described herein in a concentration described in Example 13, or in about a concentration described in Example 13. In some embodiments, the first and second surfactants are present in any composition described herein in concentrations described in Example 13, or in about concentrations described in Example 13. The first and/or second surfactants in any such embodiments may be those described in Example 13.

In a particular embodiment, the first surfactant is Poloxamer 407, and the second surfactant is Polysorbate 80 and Solutol HS-15. See, e.g., Example 14 and 27. In some embodiments, the first surfactant is present in any composition described herein in a concentration described in Example 14 or 27, or in about a concentration described in Example 14 or 27. In some embodiments, the second surfactant is present in any composition described herein in a concentration described in Example 14 or 27, or in about a concentration described in Example 14 or 27. In some embodiments, the first and second surfactants are present in any composition described herein in concentrations described in Example 14 or 27, or in about concentrations described in Example 14 or 27. The first and/or second surfactants in any such embodiments may be those described in Example 14 or 27.

In a particular embodiment, the first surfactant is Poloxamer 407 and Poloxamer 188, and the second surfactant is Polyoxyl 35 Castor Oil and Solutol HS-15. See, e.g., Example 15 and 37. In some embodiments, the first surfactant is present in any composition described herein in a concentration described in Example 15 or 37, or in about a concentration described in Example 15 or 37. In some embodiments, the second surfactant is present in any composition described herein in a concentration described in Example 15 or 37, or in about a concentration described in Example 15 or 37. In some embodiments, the first and second surfactants are present in any composition described herein in concentrations described in Example 15 or 37, or in about concentrations described in Example 15 or 37. The first and/or second surfactants in any such embodiments may be those described in Example 15 or 37.

In a particular embodiment, the first surfactant is Poloxamer 407, and the second surfactant is PEG 40 Hydrogenated Castor Oil. See, e.g., Example 22 and 28. In some embodiments, the first surfactant is present in any composition described herein in a concentration described in Example 22 or 28, or in about a concentration described in Example 22 or 28. In some embodiments, the second surfactant is present in any composition described herein in a concentration described in Example 22 or 28, or in about a concentration described in Example 22 or 28. In some embodiments, the first and second surfactants are present in any composition described herein in concentrations described in Example 22 or 28, or in about concentrations described in Example 22 or 28. The first and/or second surfactants in any such embodiments may be those described in Example 22 or 28.

In a particular embodiment, the first surfactant is Poloxamer 407, and the second surfactant is PEG 35 Castor Oil. See, e.g., Example 29. In some embodiments, the first surfactant is present in any composition described herein in a concentration described in Example 29, or in about a concentration described in Example 29. In some embodiments, the second surfactant is present in any composition described herein in a concentration described in Example 29, or in about a concentration described in Example 29. In some embodiments, the first and second surfactants are present in any composition described herein in concentrations described in Example 29, or in about concentrations described in Example 29. The first and/or second surfactants in any such embodiments may be those described in Example 29.

In a particular embodiment, the first surfactant is Poloxamer 407, and the second surfactant is Polyoxyl 35 Castor Oil and PEG 40 Hydrogenated Castor Oil. See, e.g., Example 30. In some embodiments, the first surfactant is present in any composition described herein in a concentration described in Example 30, or in about a concentration described in Example 30. In some embodiments, the second surfactant is present in any composition described herein in a concentration described in Example 30, or in about a concentration described in Example 30. In some embodiments, the first and second surfactants are present in any composition described herein in concentrations described in Example 30, or in about concentrations described in Example 30. The first and/or second surfactants in any such embodiments may be those described in Example 30.

In a particular embodiment, the first surfactant is Poloxamer 407, and the second surfactant is Polyoxyl 35 Castor Oil and Solutol HS-15. See, e.g., Example 31, 34, 35, and 36. In some embodiments, the first surfactant is present in any composition described herein in a concentration described in Example 31, 34, 35, or 36, or in about a concentration described in Example 31, 34, 35, or 36. In some embodiments, the second surfactant is present in any composition described herein in a concentration described in Example 31, 34, 35, or 36, or in about a concentration described in Example 31, 34, 35, or 36. In some embodiments, the first and second surfactants are present in any composition described herein in concentrations described in Example 31, 34, 35, or 36, or in about concentrations described in Example 31, 34, 35, or 36. The first and/or second surfactants in any such embodiments may be those described in Example 31, 34, 35, or 36.

In a particular embodiment, the first surfactant is Poloxamer 407 and Poloxamer 188, and the second surfactant is polysorbate 80. See, e.g., Example 38. In some embodiments, the first surfactant is present in any composition described herein in a concentration described in Example 38, or in about a concentration described in Example 38. In some embodiments, the second surfactant is present in any composition described herein in a concentration described in Example 38, or in about a concentration described in Example 38. In some embodiments, the first and second surfactants are present in any composition described herein in concentrations described in Example 38, or in about concentrations described in Example 38. The first and/or second surfactants in any such embodiments may be those described in Example 38.

In a particular embodiment, the first surfactant is Poloxamer 338, and the second surfactant is polysorbate 80. See, e.g., Example 41. In some embodiments, the first surfactant is present in any composition described herein in a concentration described in Example 41, or in about a concentration described in Example 41. In some embodiments, the second surfactant is present in any composition described herein in a concentration described in Example 41, or in about a concentration described in Example 41. In some embodiments, the first and second surfactants are present in any composition described herein in concentrations described in Example 41, or in about concentrations described in Example 41. The first and/or second surfactants in any such embodiments may be those described in Example 41.

C. Methods of Making

In another aspect, provided herein is a method of making an aqueous composition described herein comprising CBD. The method includes: combining CBD with a first surfactant, water, and an organic solvent to form a first composition; removing the organic solvent from the first solution to form a second composition; and adding a second surfactant to the second solution to obtain the aqueous composition.

The method described herein leads to the solubilization of CBD as a concentrated solution in water with surfactants. It is notable that merely combining CBD, a first surfactant, and a second surfactant in water, generally does not lead to solubilization of CBD, particularly not at certain concentrations described herein (see, e.g., the examples and/or the embodiments described above for exemplary concentrations). Without wishing to be bound by any particular theory, the initial dissolution in an aqueous-organic solvent mixture with the first surfactant followed by addition of the second surfactant may beneficially modulate the size of aggregates to reduce the amount of or prevent formation of relatively large aggregates, and/or provide a clear and/or stable solution. The compositions may include a low concentration of surfactant as compared to conventional preparations. It is also possible to obtain larger amounts of drug per volume in aqueous solutions than with conventional methods. This method also provides organic solvent-free pharmaceutical compositions.

In some aspects, combining CBD with a first surfactant, water, and an organic solvent is done at room temperature or at a temperature of about 5 to 10° C., 10 to 20° C., 20 to 30° C., 30 to 40° C. or 40 to 50° C. In a particular embodiment, the temperature is maintained at 25° C. In some aspects, the combining is performed optionally with agitation. In an embodiment, agitating includes stirring, shaking, sonicating, rotating, inverting, or combinations thereof. In an embodiment, the combination of CBD, a first surfactant, water, and an organic solvent is agitated for at least 5 seconds, at least 30 seconds (e.g., 30 to 60 seconds), at least 1 minute (e.g., 1 to 5 minutes), at least 5 minutes (e.g., 5 to 10 minutes), at least 10 minutes (e.g., 10 to 20 minutes), at least 20 minutes (e.g., 20 to 30 minutes), or at least 30 minutes.

In some aspects, the organic solvent is an alkanol, such as ethanol, or an alkane, such as a C₃, C₄, C₅, or C₆ alkane, such as butane. In some embodiments, the organic solvent is an alcohol, alkane (such as a C₃, C₄, C₅, or C₆ alkane, such as butane or pentane), ether, ester, ketone, or any combination thereof. In some embodiments, the organic solvent is ethanol, isopropanol, pentane, ethyl ether, acetone, ethyl acetate, or any combination thereof. In some aspects, the combining is done in an organic solvent and water at a volume ratio of from 3:1 to 1:3, or a volume ratio of 1:2, 1.4:1 to 1.8:1, or a ratio of 1.5:1 or 1.6:1. In some aspects, the concentration of CBD in the total volume of water and organic solvent is from about 1 g per 5 mL to 1 g per 15 mL. In some embodiments, the organic solvent is or comprises ethanol, and/or the organic solvent (e.g., ethanol, or any other solvent described herein) is present in a concentration of 15% to 60% by weight. In some embodiments, the concentration of the organic solvent (e.g., ethanol, or any other solvent described herein) is 15-20% by weight, 20-25% by weight, 25-30% by weight, 30-35% by weight, 35-40% by weight, 40-45% by weight, 45-50% by weight, 50-55% by weight, or 55-60% by weight.

In some aspects, the removing of the organic solvent is accomplished by distillation. Examples of distillation include rotary evaporation, distillation under reduced pressure, and distillation at atmospheric pressure, optionally with contemporaneous warming of the solution up to a temperature of about 75° C., e.g., about 60° C.

In some aspects, provided herein is a method of making an aqueous composition described herein comprising CBD. The method includes: forming a first mixture comprising CBD and a first surfactant as solids; melting the CBD and the first surfactant; and combining the first mixture with a solution comprising a second surfactant to obtain the aqueous composition. In some embodiments, melting the CBD and the first surfactant comprises heating the CBD and the first surfactant to a temperature at or above the melting point of CBD (such as 66° C.). In some embodiments, the temperature is in the range of 66° C.-100° C., 66° C.-90° C., 66° C.-80° C., 66° C.-75° C. In some embodiments, temperature is about 70° C. The first mixture may be cooled before adding the solution comprising the second surfactant, or the aqueous composition may be cooled after adding the solution comprising the second surfactant (or both). The cooling may be passive cooling. The cooling may be to room temperature or a temperature in the range of 18° C.-40° C. In some embodiments, the temperature of the first mixture is at or above 40° C., 45° C., 50° C., 55° C., 60° C., 66° C., 68° C., or 70° C. when combined with the solution comprising the second surfactant. For example, the temperature of the first mixture when combined with the solution comprising the second surfactant may be in the range of 66° C.-100° C., 66° C.-90° C., 66° C.-80° C., 66° C.-75° C. In some embodiments, the temperature is about 70° C. In some embodiments, water to the first mixture before melting. In other embodiments, water is not added to the first mixture before melting. In any of these embodiments, the method can be performed without adding organic solvent to the first mixture before melting. In some embodiments, organic solvent is not used in the method.

In some aspects, the second surfactant is added as an aqueous solution. In some aspects, the second surfactant is added as a 10, 15, or 20 wt % aqueous solution. In some aspects, addition of the second surfactant provides a clear aqueous micellar solution with micellar aggregates having a Z-average (d·nm) particle size of less than about 200 nm, e.g., 5 to 200 nm, less than about 100 nm, 5 to 100 nm, 5 to 50 nm, 9 to 35 nm, 10 to 15 nm, 15 to 20 nm, or less than about 50 nm.

In some aspects, the methods of making comprise filtering the aqueous composition or CBD concentrate. Filtration may be sterile filtration. In an embodiment, the solution may be filtered, e.g., using a 0.2 μm filter.

In some aspects, the disclosure provides a method of making a CBD diluted concentrate comprising diluting a CBD concentrate with a diluent.

D. Exemplary Methods and Uses

Provided herein are methods of treatment comprising administering a therapeutically effective amount of a pharmaceutical composition described herein to a subject in need of such treatment.

1. Subjects

The compositions and methods described herein are for use with any subject in whom CBD is effective, and who is in need of treatment for pain (such as neuropathic pain or cancer-related pain), spasticity, anxiety, cognition, movement disorders, epilepsy (including childhood epilepsy, such as Lennox-Gastaut syndrome or Dravet syndrome), or dementia (including Alzheimer's disease, Vascular Dementia, Dementia with Lewy bodies (DLB), Parkinson's disease, Frontotemporal dementia or Huntington's disease). In some embodiments, the subject is a mammal. In some embodiments, the mammal is a human. In some embodiments, the mammal is a cat. In some embodiments, the mammal is a dog. In some embodiments, the mammal is a ruminant. In some embodiments, the mammal is a horse, cow, pig, sheep, or goat.

2. Sites of Administration

The pharmaceutical compositions described herein may be administered by any mode of administration. Exemplary modes of administration include oral, parenteral, intravenous, subcutaneous, intrathecal, or inhalation, or administration by absorption, e.g., through the skin or buccal surface.

In some embodiments, the pharmaceutical composition is administered by injection. Injections may be performed, e.g., using a 1 cc syringe, or more generally, a size of syringe appropriate for the dosage volume.

In an embodiment, the pharmaceutical compositions described herein are formulated for parenteral administration, e.g., intravenous or intramuscular administration.

3. Dosage

In some embodiments, the pharmaceutical composition comprising CBD is administered at a daily dose of 200 mg to 3 g, in a single or divided dose. In some embodiments, the dose of CBD ranges from 250 mg to 1.5 g. For example, e.g., in humans, a total dose of CBD is 300 mg to 1.5 g per day, or 300 mg to 1.2 g per day, or 200 mg to 600 mg per individual dose, administered once or twice or more times daily.

### IV. EXAMPLES

### 1. Poloxamer 407 and PEG 40 Hydrogenated Castor Oil

First surfactant: Poloxamer 407
Second surfactant: PEG 40 hydrogenated castor oil (10% wt in water)
Procedure: CBD powder (3.0 g) and Poloxamer 407 (3.75 g) were combined in a mixture of 15 mL ethanol and 10 mL water, and the resulting mixture was stirred to form a clear colorless or pink solution. The solution was concentrated on a rotary evaporator to remove ethanol. PEG 40 hydrogenated castor oil (25 g of 10 wt % solution in water) was added, and the resulting mixture was stirred at 700 rpm to generate a colorless or light pink, clear solution. The solution was filtered under sterile conditions with a 0.2 μm filter. The product was a 5.7% by weight CBD solution. CBD concentrations described in this and subsequent examples are generally accurate to within 10% of the stated value.

### 2. Poloxamer 407 and Solutol HS-15

First surfactant: Poloxamer 407
Second surfactant: Solutol HS-15 (20% wt in water)
Procedure: CBD powder (2.5 g) and Poloxamer 407 (2.5 g) were combined in a mixture of 17 mL ethanol and 10 mL water, and the resulting mixture was stirred to form a clear colorless or pink solution. The solution was distilled at 60° C. under vacuum to remove ethanol. Solutol HS-15 (30 g of 20 wt % solution in water) was added, and the resulting mixture was stirred at 700 rpm to generate a colorless or light pink, clear solution. The solution was filtered under sterile conditions with a 0.2 μm filter. The product was a 5.0% by weight CBD solution.

### 3. Poloxamer 407 and PEG 40 Hydrogenated Castor Oil

First surfactant: Poloxamer 407
Second surfactant: PEG 40 hydrogenated castor oil (20% wt in water)
Procedure: CBD powder (2.75 g) and Poloxamer 407 (2.25 g) were combined in a mixture of 15 mL ethanol and 10 mL water, and the resulting mixture was stirred to form a clear colorless or pink solution. The solution was distilled at 60° C. under vacuum to remove ethanol. PEG 40 hydrogenated castor oil (25 g of 20 wt % solution in water) was added, and the resulting mixture was stirred at 700 rpm to generate a colorless or light pink clear solution. The solution was filtered under sterile conditions with a 0.2 μm filter. The product was a 6.6% by weight CBD solution.

### 4. Poloxamer 407 and PEG 35 Castor Oil

First surfactant: Poloxamer 407
Second surfactant: PEG 35 castor oil (15% wt in water)
Procedure: CBD powder (2.75 g) and Poloxamer 407 (2.25 g) were combined in a mixture of 15 mL ethanol and 10 mL water, and the resulting mixture was stirred to form a clear colorless or pink solution. The solution was distilled at 60° C. under vacuum to remove ethanol. PEG 35 castor oil (30 g of 15 wt % solution in water) was added, and the resulting mixture was stirred at 700 rpm to generate a colorless or light pink clear solution. The solution was filtered under sterile conditions with a 0.2 μm filter. The product was a 5.8% by weight CBD solution.

### 5. Poloxamer 407 and Polyoxyl 35 Castor Oil/PEG 40 Hydrogenated Castor Oil

First surfactant: Poloxamer 407
Second surfactant: Polyoxyl 35 castor oil (15% wt in water) and PEG 40 hydrogenated castor oil (15% wt in water)
Procedure: CBD powder (2.75 g) and Poloxamer 407 (2.50 g) were combined in a mixture of 15 mL ethanol and 10 mL water, and the resulting mixture was stirred to form a clear colorless or pink solution. The solution was distilled at 60° C. under vacuum to remove ethanol. Polyoxyl 35 castor oil (15 g) and PEG 40 hydrogenated castor oil (15 g) were added, and the resulting mixture was stirred at 700 rpm to generate a colorless or light pink clear solution. The solution was filtered under sterile conditions with a 0.2 μm filter. The product was a 6.2% by weight CBD solution.

### 6. Poloxamer 407 and Polyoxyl 35 Castor Oil/Solutol HS-15

First surfactant: Poloxamer 407
Second surfactant: Polyoxyl 35 castor oil (15% wt in water) and Solutol HS-15 (15% wt in water)
Procedure: CBD powder (2.75 g) and Poloxamer 407 (2.50 g) were combined in a mixture of 15 mL ethanol and 10 mL water, and the resulting mixture was stirred to form a clear colorless or pink solution. The solution was distilled at 60° C. under vacuum to remove ethanol. Polyoxyl 35 castor oil and Solutol HS-15 (combined 30 g of 15 wt % solutions in water total per table below) were added and each resulting mixture was stirred at 700 rpm to generate a colorless or light pink, clear solution. The solutions were filtered under sterile conditions with a 0.2 μm filter.

| | Polyoxyl 35 Castor Oil | Solutol HS-15 | CBD |
|---|---|---|---|
| | (g 15 wt % | (g 15 wt % | wt % |
| Ex. | solution in water) | solution in water) | (final) |
| 6A | 10 | 20 | 6.2 |
| 6B | 15 | 15 | 6.3 |
| 6C | 5 | 25 | 6.1 |

### 7. Poloxamer 407 and Polyoxyl 35 Castor Oil/Solutol HS-15

First surfactant: Poloxamer 407
Second surfactant: Polyoxyl 35 castor oil (15% wt in water) and Solutol HS-15 (15% wt in water)
Procedure: CBD powder (2.75 g) and Poloxamer 407 (mass per table below) were combined in a mixture of 15 mL ethanol and 10 mL water, and each resulting mixture was stirred to form a clear colorless or pink solution. The solutions were distilled at 60° C. under vacuum to remove ethanol. Polyoxyl 35 castor oil (15 g of 15 wt % solution in water) and Solutol HS-15 (15 g of 15 wt % solution in water) were added to each sample, and the resulting mixtures were stirred at 700 rpm to generate colorless or light pink clear solutions. The solutions were filtered under sterile conditions with a 0.2 μm filter.

| | Poloxamer | CBD wt % |
|---|---|---|
| Ex. | 407 (g) | (final) |
| 7A | 2.25 | 6.0 |
| 7B | 2.0 | 6.1 |
| 7C | 1.75 | 6.0 |

### 8. Poloxamer 407/Poloxamer 188 and Polyoxyl 35 Castor Oil/Solutol HS-15

First Surfactant: Poloxamer 407 and Poloxamer 188
Second Surfactant: Polyoxyl 35 castor oil (15% wt in water) and Solutol HS-15 (15% wt in water)
Procedure: CBD powder (2.75 g), Poloxamer 407 (1.75 g), and Poloxamer 188 (0.5 g) were combined in a mixture of 16 mL ethanol and 10 mL water, and the resulting mixture was stirred to form a colorless or light pink clear solution. The solution was distilled at 60° C. under vacuum to remove ethanol. Polyoxyl 35 castor oil (15 g of 15 wt % solution in water) and Solutol HS-15 (15 g of 15 wt % solution in water) were added and the resulting mixture was stirred at 700 rpm to generate a colorless or light pink clear solution. The solution was filtered under sterile conditions with a 0.2 μm filter. The product was a 6.0% by weight CBD solution.

### 9. Example 9

### Dilution of CBD Concentrate in DPBS

An aqueous composition comprising 5.0% CBD (50 mg/mL) prepared as described herein was diluted 1:50 with Dulbecco's phosphate buffered saline (DPBS) to produce a CBD diluted concentrate (0.1% CBD, 1.0 mg/mL). The diluted composition had a clear appearance and a Z-average particle size of 35 nm.

### 10. Example 10

### Dilution of CBD Concentrate in Lemon-Lime Carbonated Soda

An aqueous composition comprising 5.0% CBD (50 mg/mL) prepared as described herein was diluted 0.003:1 with SPRITE™ lemon-line carbonated soda beverage to provide a 0.015% CBD solution (150 mg/liter). The diluted composition had a clear appearance.

### 11. Poloxamer 407 and Polysorbate 80; Comparison to Process without Organic Solvent

First surfactant: Poloxamer 407
Second surfactant: Polysorbate 80 (20% wt in water)
Procedure: CBD powder (2.75 g) and Poloxamer 407 (2.25 g) were combined in 15 mL ethanol and 10 mL water, and the resulting mixture was stirred to form a clear colorless or pink solution. The solution was distilled at 60° C. under vacuum to remove ethanol. Polysorbate 80 (30 g of 20 wt % solution in water) was added, and the resulting mixture was stirred at 700 rpm to generate a clear or light pink solution. The solution was filtered under sterile conditions with a 0.2 μm filter. The product was a 6.0% by weight CBD solution. The duration of this process was about 4 hours.

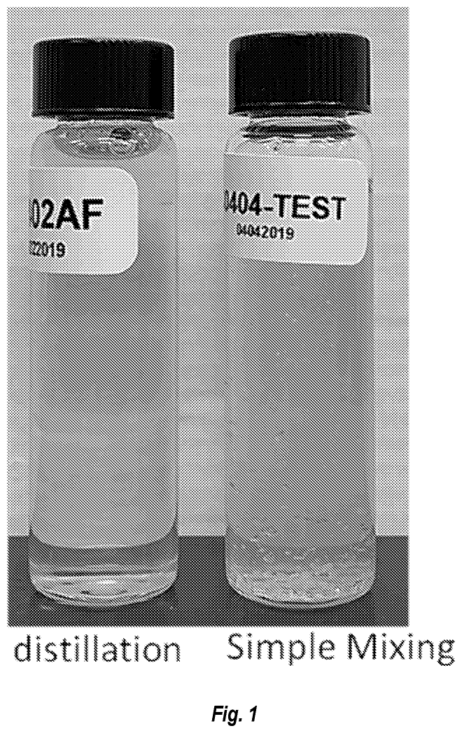

A comparative composition was prepared by mixing CBD powder (2.75 g) and Poloxamer 407 (2.25 g) in 10 mL water, and polysorbate 80 (30 g of 20 wt % solution in water) was added. The resulting mixture was stirred for 16 hours at 1000 rpm. Many visible crystals of undissolved CBD remained in the composition. See FIG. 1.

### 12. Poloxamer 407 and Vitamin E TPGS

First surfactant: Poloxamer 407
Second surfactant: Vitamin E TPGS (20% wt in water)
Procedure: CBD powder (2.75 g) and Poloxamer 407 (2.25 g) were combined in 15 mL ethanol and 10 mL water, and the resulting mixture was stirred to form a clear colorless or pink solution. The solution was distilled at 60° C. under vacuum to remove ethanol. Vitamin E TPGS (30 g of 20 wt % solution in water) was added, and the resulting mixture was stirred at 700 rpm to generate a clear or light pink solution. The solution was filtered under sterile conditions with a 0.2 μm filter. The product was a 6.0% by weight CBD solution.

### 13. Poloxamer 407 and Polysorbate 80/Vitamin E TPGS

First surfactant: Poloxamer 407
Second surfactant: Polysorbate 80 (10% wt in water) and Vitamin E TPGS (20% wt in water)
Procedure: CBD powder (2.75 g) and Poloxamer 407 (2.25 g) were combined in 15 mL ethanol and 10 mL water, and the resulting mixture was stirred to form a clear colorless or pink solution. The solution was distilled at 60° C. under vacuum to remove ethanol. Polysorbate 80 (15 g of 10 wt % solution in water) and Vitamin E TPGS (15g of 20 wt % solution in water) was added, and the resulting mixture was stirred at 700 rpm to generate a clear or light pink solution. The solution was filtered under sterile conditions with a 0.2 μm filter. The product was a 6.2% by weight CBD solution.

### 14. Poloxamer 407 and Polysorbate 80/Solutol HS-15

First surfactant: Poloxamer 407
Second surfactant: Polysorbate 80 (15% wt in water) and Solutol HS-15 (15% wt in water)
Procedure: CBD powder (2.75 g) and Poloxamer 407 (2.50 g) were combined in 15 mL ethanol and 10 mL water, and the resulting mixture was stirred to form a clear colorless or pink solution. The solution was distilled at 60° C. under vacuum to remove ethanol. Polysorbate 80 (15 g of 15 wt % solution in water) and Solutol HS-15 (15 g of 15 wt % solution in water) was added, and the resulting mixture was stirred at 700 rpm to generate a clear or light pink solution. The solution was filtered under sterile conditions with a 0.2 μm filter. The product was a 6.2% by weight CBD solution.

### 15. Poloxamer 407/Poloxamer 188 and Polyoxyl 35 Castor Oil/Solutol HS-15

First surfactant: Poloxamer 407 and Poloxamer 188
Second surfactant: Polyoxyl 35 Castor Oil (20 wt % in water) and Solutol HS-15 (20 wt % in water)
Procedure: CBD powder (4.0 g), Poloxamer 407 (1.50 g), and Poloxamer 188 (0.75 g) were combined in 19 mL ethanol and 10 mL water, and the resulting mixture was stirred to form a clear colorless or pink solution. The solution was distilled at 60° C. under vacuum to remove ethanol. Polyoxyl 35 Castor Oil (15 g of 20 wt % solution in water) and Solutol HS-15 (15 g of 20 wt % solution in water) was added, and the resulting mixture was stirred at 700 rpm to generate a colorless or light pink solution. The solution was filtered under sterile conditions with a 0.2 μm filter. The product was a 10% by weight CBD solution.

### 16. Further Example with Poloxamer 407 and PS80 with Ethanol Removed by Distillation

First surfactant: Poloxamer 407
Second surfactant: Polysorbate 80 (15% wt in water)
Procedure: CBD powder (3.0 g) and Poloxamer 407 (1.75 g) were combined in 6.44 mL ethanol and 7.63 mL water, and the resulting mixture formed an off-white or pink slurry. The mixture was distilled at 60° C. under vacuum to remove ethanol, giving an off-white slurry. Polysorbate 80 (PS80) (37.4 g of 15 wt % solution in water) was added, and the resulting mixture was stirred at 500 rpm to generate a clear colorless or light pink solution. Water was added q.s. to 50 mL total volume if needed and the product was a 6.0% by weight CBD solution.

### 17. Poloxamer 407 and PS80 with Pentane Removed by Distillation

First surfactant: Poloxamer 407
Second surfactant: Polysorbate 80 (15% wt in water)
Procedure: CBD powder (3.0 g) and Poloxamer 407 (1.75 g) were combined in 9 mL pentane and 7.63 mL water, and the resulting mixture formed an off-white or pink slurry. The mixture was distilled at 40° C. under vacuum to remove pentane, giving a paste-like off-white solution. Polysorbate 80 (PS80) (37.4 g of 15 wt % solution in water) was added, and the resulting mixture was stirred at 500 rpm to generate a clear colorless or light pink solution. Water was added q.s. to 50 mL total volume if needed and the product was a 6.0% by weight CBD solution.

### 18. Poloxamer 407 and PS80 with Ethyl Ether Removed by Distillation

First surfactant: Poloxamer 407
Second surfactant: Polysorbate 80 (15% wt in water)
Procedure: CBD powder (3.0 g) and Poloxamer 407 (1.75 g) were combined in 9 mL ethyl ether and 7.63 mL water, and the resulting mixture formed an off-white or pink slurry. The mixture was distilled at 40° C. under vacuum to remove ethyl ether, giving a paste-like off-white slurry. Polysorbate 80 (PS80) (37.4 g of 15 wt % solution in water) was added, and the resulting mixture was stirred at 500 rpm to generate a clear colorless or light pink solution. Water was added q.s. to 50 mL total volume and the product was a 6.0% by weight CBD solution.

### 19. Poloxamer 407 and PS80 with Acetone Removed by Distillation

First surfactant: Poloxamer 407
Second surfactant: Polysorbate 80 (15% wt in water)
Procedure: CBD powder (3.0 g) and Poloxamer 407 (1.75 g) were combined in 9 mL acetone and 7.63 mL water, and the resulting mixture formed an off-white or pink slurry. The mixture was distilled at 60° C. under vacuum to remove acetone, giving a paste-like off-white slurry. Polysorbate 80 (PS80) (37.4 g of 15 wt % solution in water) was added, and the resulting mixture was stirred at 500 rpm to generate a clear to semitransparent colorless or light pink solution. Water was added q.s. to 50 mL total volume if needed and the product was a 6.0% by weight CBD solution.

### 20. Poloxamer 407 and PS80 with Isopropanol Removed by Distillation

First surfactant: Poloxamer 407
Second surfactant: Polysorbate 80 (15% wt in water)
Procedure: CBD powder (3.0 g) and Poloxamer 407 (1.75 g) were combined in 9 mL isopropanol and 7.63 mL water, and the resulting mixture formed an off-white or pink slurry. The mixture was distilled at 65° C. under vacuum to remove isopropanol, giving a paste-like off-white slurry. Polysorbate 80 (PS80) (37.4 g of 15 wt % solution in water) was added, and the resulting mixture was stirred at 500 rpm to generate a clear colorless or light pink solution. Water was added q.s. to 50 mL total volume if needed and the product was a 6.0% by weight CBD solution.

### 21. Poloxamer 407 and PS80 with Ethyl Acetate Removed by Distillation

First surfactant: Poloxamer 407
Second surfactant: Polysorbate 80 (15% wt in water)
Procedure: CBD powder (3.0 g) and Poloxamer 407 (1.75 g) were combined in 9 mL ethyl acetate and 7.63 mL water, and the resulting mixture formed an off-white or pink slurry. The mixture was distilled at 60° C. to remove isopropanol, giving a paste-like off-white slurry. Polysorbate 80 (PS80) (37.4 g of 15 wt % solution in water) was added, and the resulting mixture was stirred at 500 rpm to generate a semitransparent colorless or light pink solution. Water was added q.s. to 50 mL total volume if needed and the product was a 6.0% by weight CBD solution.

### 22. Poloxamer 407 and PEG 40 Hydrogenated Castor Oil with Ethanol Removed by Rotary Evaporation

First surfactant: Poloxamer 407
Second surfactant: PEG 40 Hydrogenated Castor Oil (10% wt in water)
Procedure: CBD powder (3.0 g) and Poloxamer 407 (3.75 g) were combined in 5 mL ethanol and 10 mL water, and the resulting mixture formed a clear colorless or pink solution. Ethanol was removed by rotary evaporation. PEG 40 Hydrogenated Castor Oil (25 g of 10 wt % solution in water) was added, and the resulting mixture was stirred at 700 rpm to generate a colorless or light pink clear solution. The solution was sterile-filtered through a 0.2 μm filter. Water was added q.s. to 50 mL total volume if needed and the product was a 6.0% by weight CBD solution.

### 23. Poloxamer 407 and PS80 with water and 70° C. Heating and Cooling Before PS80 Addition

First surfactant: Poloxamer 407
Second surfactant: Polysorbate 80 (15% wt in water)
Procedure: CBD powder (3.0 g) and Poloxamer 407 (1.75 g) were combined in 8.25 mL water, and heated with stirring until all solids melted. The mixture was passively cooled to room temperature with stirring (300 rpm). PS80 (37 g of 15 wt % solution in water) was added, and the resulting mixture was stirred at 500 rpm to generate a colorless or light pink clear solution. The product was a 6.0% by weight CBD solution.

Alternative Procedure: CBD powder (3.0 g) and Poloxamer 407 (1.75 g) were heated to 70° C. with stirring until all solids melted. The mixture was passively cooled to room temperature with stirring (300 rpm). PS80 (37 g of 15 wt % solution in water) and water (8.25 g) were added, and the resulting mixture was stirred at 500 rpm to generate a clear colorless or light pink clear solution. Water was added q.s. to 50 mL total volume if needed and the product was a 6.0% by weight CBD solution.

### 24. Poloxamer 407 and PS80 with water and 70° C. Heating and Cooling After PS80 Addition

First surfactant: Poloxamer 407
Second surfactant: Polysorbate 80 (15% wt in water)
Procedure: CBD powder (3.0 g) and Poloxamer 407 (1.75 g) were combined in 8.25 mL water, and heated with stirring (300 rpm) until all solids melted. PS80 (37 g of 15 wt % solution in water) was added, and the resulting mixture was passively cooled to room temperature with stirring at 500 rpm to generate a colorless or light pink clear solution. The product was a 6.0% by weight CBD solution.

Alternative Procedure: CBD powder (3.0 g) and Poloxamer 407 (1.75 g) were heated to 70° C. with stirring (300 rpm) until all solids melted. PS80 (37 g of 15 wt % solution in water) and water (8.25 g) were added with continued stirring (300 rpm), and the resulting mixture was passively cooled to room temperature with stirring at 500 rpm to generate a colorless or light pink clear solution. Water was added q.s. to 50 mL total volume if needed and the product was a 6.0% by weight CBD solution.

### 25. Poloxamer 407 and PS80 with Water Added Before 70° C. Heating and Cooling

First surfactant: Poloxamer 407
Second surfactant: Polysorbate 80 (15 wt %)
Procedure: CBD powder (3.0 g) and Poloxamer 407 (1.75 g) were combined with 8.25 mL water and PS80 37 g of 15 wt % solution in water), and heated to 70° C. with stirring (300 rpm) until all solids melted. The resulting mixture was passively cooled to room temperature with stirring at 500 rpm to generate a milky white or light pink clear solution. Water was added q.s. to 50 mL total volume if needed and the product was a 6.0% by weight CBD solution.

### 26. Poloxamer 407 and PS80 with Water and Stirring at Room Temperature

First surfactant: Poloxamer 407
Second surfactant: Polysorbate 80 (15 wt %)
Procedure: CBD powder (3.0 g) was combined with Poloxamer 407 (8.81 g of 20 wt % solution in water), PS80 (37 g of 15 wt % solution in water), and water (1 g) and stirred at 500 rpm at room temperature for 6 days, giving a pale semitransparent to milky 6.0% by weight solution containing large undissolved particles. Large undissolved particles were removed by filtration with a 0.2 μM syringe filter.

### 27. Poloxamer 407 and Solutol HS-15 with Ethanol/Water Mixture and Distillation

First surfactant: Poloxamer 407
Second surfactant: Solutol HS-15 (20% wt in water)
Procedure: CBD powder (2.5 g) and Poloxamer 407 (2.5 g) were combined in 27 mL of a 1.7:1 (by volume) ethanol:water mixture, giving a clear colorless or pink solution. Ethanol was removed by distillation at 60° C. Solutol HS-15 (30 g of 20 wt % solution in water) was added, and the resulting mixture was stirred at 700 rpm. The solution remained colorless or light pink and clear. Water was added if needed and the product was a 6.0% by weight CBD solution. The solution was sterile-filtered through a 0.2 μm filter.

### 28. Poloxamer 407 and PEG 40 Hydrogenated Castor Oil with Ethanol Removed by Distillation

First surfactant: Poloxamer 407
Second surfactant: PEG 40 Hydrogenated Castor Oil (20% wt in water)
Procedure: CBD powder (2.75 g) and Poloxamer 407 (2.25 g) were combined in 25 mL of an ethanol/water mixture (1.5:1 by volume), and the resulting mixture formed a clear colorless or pink solution. Ethanol was removed by distillation at 60° C. PEG 40 Hydrogenated Castor Oil (25 g of 20 wt % solution in water) was added, and the resulting mixture was stirred at 700 rpm to generate a colorless or light pink clear solution. The solution was sterile-filtered through a 0.2 um filter. Water was added if needed and the product was a 7.0% by weight CBD solution.

### 29. Poloxamer 407 and PEG 35 Castor Oil with Ethanol Removed by Distillation

First surfactant: Poloxamer 407
Second surfactant: PEG 35 Castor Oil (15% wt in water)
Procedure: CBD powder (2.75 g) and Poloxamer 407 (2.25 g) were combined in 25 mL of an ethanol/water mixture (1.5:1 by volume), and the resulting mixture formed a clear colorless or pink solution. Ethanol was removed by distillation at 60° C. PEG 35 Castor Oil (30 g of 15 wt % solution in water) was added, and the resulting mixture was stirred at 700 rpm to generate a colorless or light pink clear solution. The solution was sterile-filtered through a 0.2 μm filter. Water was added if needed and the product was a 6.0% by weight CBD solution.

### 30. Poloxamer 407, Polyoxyl 35 Castor Oil and PEG 40 Hydrogenated Castor Oil with Ethanol Removed by Distillation

First surfactant: Poloxamer 407
Second surfactant: Polyoxyl 35 Castor Oil (15% wt in water) and PEG 40 Hydrogenated Castor Oil (15% wt in water)
Procedure: CBD powder (2.75 g) and Poloxamer 407 (2.50 g) were combined in 25 mL of an ethanol/water mixture (1.5:1 by volume), and the resulting mixture formed a clear colorless or pink solution. Ethanol was removed by distillation at 60° C. Polyoxyl 35 Castor Oil (15 g of a 15 wt % solution in water) and PEG 40 Hydrogenated Castor Oil (15 g of a 15 wt % solution in water) were added, and the resulting mixture was stirred at 700 rpm to generate a colorless or light pink clear solution. The solution was sterile-filtered through a 0.2 μm filter. Water was added if needed and the product was a 6.0% by weight CBD solution.

### 31. Poloxamer 407, Polyoxyl 35 Castor Oil and Solutol HS-15 with Ethanol Removed by Distillation

First surfactant: Poloxamer 407
Second surfactant: Polyoxyl 35 Castor Oil (15% wt in water) and Solutol HS-15 (15% wt in water)
Procedure: CBD powder (2.75 g) and Poloxamer 407 (2.50 g) were combined in 25 mL of an ethanol/water mixture (1.5:1 by volume), and the resulting mixture formed a clear colorless or pink solution. Ethanol was removed by distillation at 60° C. under vacuum. Polyoxyl 35 Castor Oil (15 g of a 15 wt % solution in water) and Solutol HS-15 (15 g of a 15 wt % solution in water) were added, and the resulting mixture was stirred at 700 rpm to generate a colorless or light pink clear solution. The solution was sterile-filtered through a 0.2 μm filter. Water was added if needed and the product was a 6.0% by weight CBD solution.

### 32. Poloxamer 407 and Polysorbate 80 with Ethanol Removed by Distillation

First surfactant: Poloxamer 407
Second surfactant: Polysorbate 80 (20% wt in water)
Procedure: CBD powder (2.75 g) and Poloxamer 407 (2.25 g) were combined in 25 mL of an ethanol/water mixture (1.5:1 by volume), and the resulting mixture formed a clear colorless or pink solution. Ethanol was removed by distillation at 60° C. under vacuum. Polysorbate 80 (30 g of a 20 wt % solution in water) was added, and the resulting mixture was stirred at 700 rpm to generate a colorless or light pink clear solution. The solution was sterile-filtered through a 0.2 μm filter. Water was added if needed and the product was a 6.0% by weight CBD solution.

### 33. Poloxamer 407 and Vitamin E TPGS with Ethanol Removed by Distillation

First surfactant: Poloxamer 407
Second surfactant: Vitamin E TPGS (20% wt in water)
Procedure: CBD powder (2.75 g) and Poloxamer 407 (2.25 g) were combined in 25 mL of an ethanol/water mixture (1.5:1 by volume), and the resulting mixture formed a clear colorless or pink solution. Ethanol was removed by distillation at 60° C. Vitamin E TPGS (30 g of a 20 wt % solution in water) was added, and the resulting mixture was stirred at 700 rpm to generate a colorless or light pink clear solution. The solution was sterile-filtered through a 0.2 μm filter. Water was added if needed and the product was a 6.0% by weight CBD solution.

### 34. Poloxamer 407, Polyoxyl 35 Castor Oil and Solutol HS-15 with Ethanol Removed by Distillation

First surfactant: Poloxamer 407
Second surfactant: Polyoxyl 35 Castor Oil (15% wt in water) and Solutol HS-15 (15% wt in water)
Procedure: CBD powder (2.75 g) and Poloxamer 407 (1.75 g) were combined in 25 mL of an ethanol/water mixture (1.5:1 by volume), and the resulting mixture formed a clear colorless or pink solution. Ethanol was removed by distillation at 60° C. Polyoxyl 35 Castor Oil (15 g of a 15 wt % solution in water) and Solutol HS-15 (15 g of a 15 wt % solution in water) were added, and the resulting mixture was stirred at 700 rpm to generate a colorless or light pink clear solution. The solution was sterile-filtered through a 0.2 μm filter. Water was added if needed and the product was a 6.0% by weight CBD solution.

### 35. Poloxamer 407, Polyoxyl 35 Castor Oil and Solutol HS-15 with Ethanol Removed by Distillation

First surfactant: Poloxamer 407
Second surfactant: Polyoxyl 35 Castor Oil (15% wt in water) and Solutol HS-15 (15% wt in water)
Procedure: CBD powder (2.75 g) and Poloxamer 407 (2.0 g) were combined in 25 mL of an ethanol/water mixture (1.5:1 by volume), and the resulting mixture formed a clear colorless or pink solution. Ethanol was removed by distillation at 60° C. Polyoxyl 35 Castor Oil (15 g of a 15 wt % solution in water) and Solutol HS-15 (15 g of a 15 wt % solution in water) were added, and the resulting mixture was stirred at 700 rpm to generate a colorless or light pink clear solution. The solution was sterile-filtered through a 0.2 μm filter. Water was added if needed and the product was a 6.1% by weight CBD solution.

### 36. Poloxamer 407, Polyoxyl 35 Castor Oil and Solutol HS-15 with Ethanol Removed by Distillation

First surfactant: Poloxamer 407
Second surfactant: Polyoxyl 35 Castor Oil (15% wt in water) and Solutol HS-15 (15% wt in water)
Procedure: CBD powder (2.75 g) and Poloxamer 407 (2.25 g) were combined in 25 mL of an ethanol/water mixture (1.5:1 by volume), and the resulting mixture formed a clear colorless or pink solution. Ethanol was removed by distillation at 60° C. Polyoxyl 35 Castor Oil (15 g of a 15 wt % solution in water) and Solutol HS-15 (15 g of a 15 wt % solution in water) were added, and the resulting mixture was stirred at 700 rpm to generate a colorless or light pink clear solution. The solution was sterile-filtered through a 0.2 μm filter. Water was added if needed and the product was a 6.0% by weight CBD solution.

### 37. Poloxamer 407, Poloxamer 188, Polyoxyl 35 Castor Oil and Solutol HS-15 with Ethanol Removed by Distillation

First surfactant: Poloxamer 407 and Poloxamer 188
Second surfactant: Polyoxyl 35 Castor Oil (15% wt in water) and Solutol HS-15 (15% wt in water)
Procedure: CBD powder (2.75 g), Poloxamer 407 (1.75 g) and Poloxamer 188 (0.5 g) were combined in 26 mL of an ethanol/water mixture (1.6:1 by volume), and the resulting mixture formed a clear colorless or pink solution. Ethanol was removed by distillation at 60° C. Polyoxyl 35 Castor Oil (15 g of a 15 wt % solution in water) and Solutol HS-15 (15 g of a 15 wt % solution in water) were added, and the resulting mixture was stirred at 700 rpm to generate a colorless or light pink clear solution. The solution was sterile-filtered through a 0.2 μm filter. Water was added if needed and the product was a 6.0% by weight CBD solution.

### 38. Poloxamer 407, Poloxamer 188, and PS80 with Ethanol Removed by Distillation

First surfactant: Poloxamer 407 and Poloxamer 188
Second surfactant: Polysorbate 80 (15% wt in water)
Procedure: CBD powder (2.75 g), Poloxamer 407 (1.75 g) and Poloxamer 188 (0.5 g) were combined in 26 mL of an ethanol/water mixture (1.6:1 by volume), and the resulting mixture formed a clear colorless or pink solution. Ethanol was removed by distillation at 60° C. Polysorbate 80 (30 g of a 15 wt % solution in water) was added, and the resulting mixture was stirred at 700 rpm to generate a colorless or light pink clear solution. The solution was sterile-filtered through a 0.2 μm filter. Water was added if needed and the product was a 6.0% by weight CBD solution.

### 39. Poloxamer 407, Poloxamer 188, Polyoxyl 35 Castor Oil and Solutol HS-15 with Ethanol Removed by Distillation

First surfactant: Poloxamer 407 and Poloxamer 188
Second surfactant: Polyoxyl 35 Castor Oil (20% wt in water) and Solutol HS-15 (20% wt in water)
Procedure: CBD powder (4.0 g) and Poloxamer 407 (1.50 g) were combined in 29 mL of an ethanol/water mixture (1.9:1 by volume), and the resulting mixture formed a clear colorless or pink solution. Ethanol was removed by distillation at 60° C. Polyoxyl 35 Castor Oil (15 g of a 20 wt % solution in water) and Solutol HS-15 (15 g of a 20 wt % solution in water) were added, and the resulting mixture was stirred at 700 rpm to generate a colorless or light pink clear solution. The solution was sterile-filtered through a 0.2 μm filter. Water was added if needed and the product was a 10.0% by weight CBD solution.

### 40. Poloxamer 407 and Polysorbate 80 with Citrate Buffer and with Ethanol Removed by Distillation

First surfactant: Poloxamer 407
Second surfactant: Polysorbate 80 (15% wt in 20 mM citrate buffer pH 4.0)
Procedure: CBD powder (3.0 g) and Poloxamer 407 (1.75 g) were combined an ethanol/citrate buffer mixture (9 mL of ethanol and 7.63 mL of 20 mM citrate buffer pH 4.0), and the resulting mixture formed a off-white or pink slurry. Ethanol was removed by distillation at 60° C. under vacuum and the resulting mixture formed an off-white solution. Polysorbate 80 (37.4 g of a 15 wt % solution in 20 mM citrate buffer pH 4.0) was added, and the resulting mixture was stirred at 500 rpm to generate a colorless or light pink clear solution. Water was added if needed and the product was a 6.0% by weight CBD solution.

### 41. Poloxamer 407 and Polysorbate 80 with Phosphate Buffer and with Ethanol Removed by Distillation

First surfactant: Poloxamer 407
Second surfactant: Polysorbate 80 (15% wt in 20 mM citrate buffer pH 4.0)
Procedure: CBD powder (3.0 g) and Poloxamer 407 (1.75 g) were combined an ethanol/citrate buffer mixture (9 mL of 100% ethanol and 7.63 mL of 20 mM phosphate buffer pH 4.0), and the resulting mixture formed a off-white or pink slurry. Ethanol was removed by distillation at 60° C. under vacuum and the resulting mixture formed an off-white solution. Polysorbate 80 (37.4 g of a 15 wt % solution in 20 mM phosphate buffer pH 4.0) was added, and the resulting mixture was stirred at 500 rpm to generate a colorless or light pink clear to semitransparent solution. Water was added if needed and the product was a 6.0% by weight CBD solution.

### 42. Poloxamer 338 and Polysorbate 80 with Phosphate Buffer and with Ethanol Removed by Distillation

First surfactant: Poloxamer 338
Second surfactant: Polysorbate 80 (15% wt in water)
Procedure: CBD powder (2.75 g) and Poloxamer 338 (1.85 g) were combined an ethanol/water mixture (20 mL of 1:1 mixture by volume), and the resulting mixture formed a colorless or pink solution. Ethanol was removed by distillation at 60° C. under vacuum. Polysorbate 80 (30 g of a 15 wt % solution in water) was added, and the resulting mixture was stirred at 700 rpm to generate a colorless or light pink clear solution. The solution was sterile-filtered with a 0.2 μm filter. Water was added if needed and the product was a 6.0% by weight CBD solution.

### 43. Stability Study of CBD Formulations Using Citric Buffer

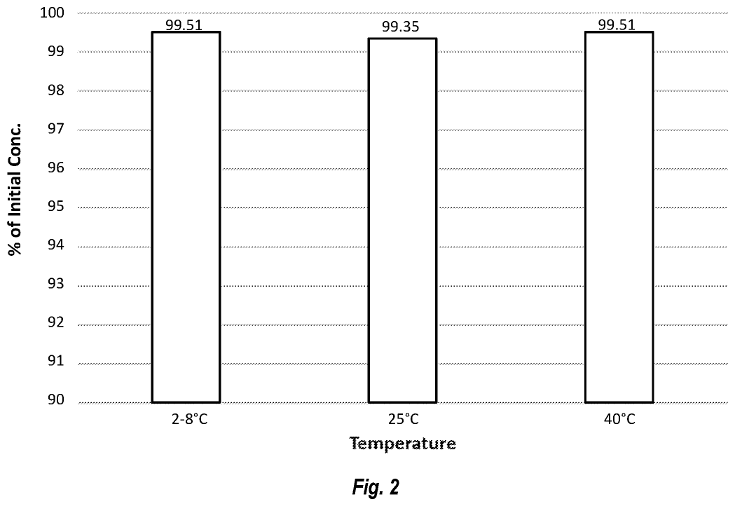

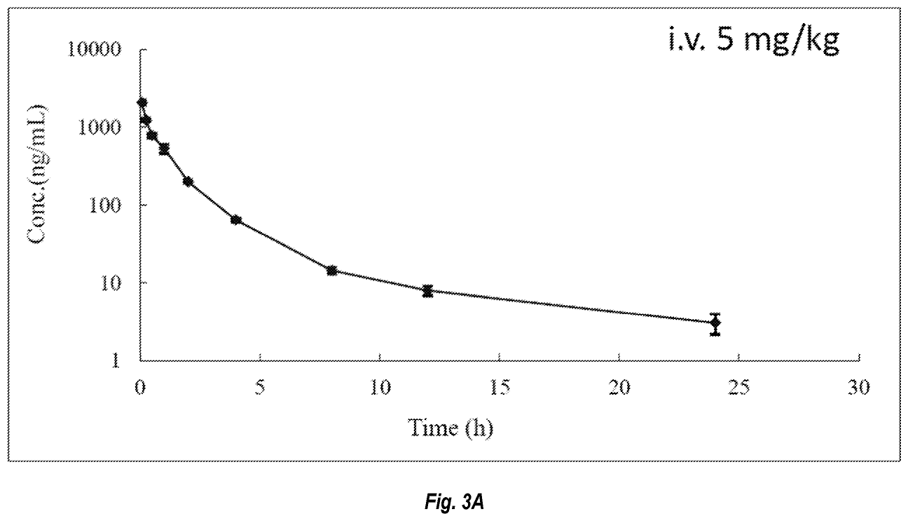

The stability of CBD in citric buffer over a three-month time period was studied following guidance provided by the Q1A (R2) document (on Stability Testing of New Drug Substances and Products) put forth by the International Council for Harmonisation of Technical Requirements for Pharmaceuticals for Human Use (ICH).
Procedure: Three samples of CBD in citric buffer formulations, prepared as described in example 40 above, were incubated separately at three different conditions: (1) 2° C. to 8° C., (2) 25° C. with 60% relative humidity, and (3) 40° C. with 75% relative humidity. After three months, CBD concentration was measured for each of the samples: (1) The sample stored at 2° C. to 8° C. had 99.51% of its initial concentration; (2) the sample stored at 25° C. with 60% relative humidity had 99.35% of its initial concentration; and (3) the sample stored at 40° C. with 75% to 8° C. had 99.35% of its initial concentration (FIG. 2).

### 44. Pharmacokinetics Following Intravenous Administration

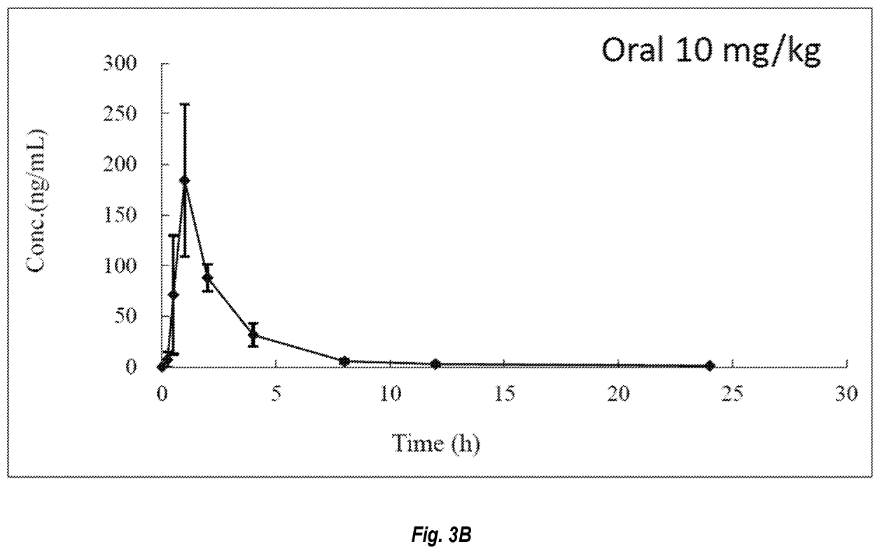

The formulation prepared in Example 16 was diluted to 10 mg/ml in 10 mM phosphate-buffered saline, filtered with an 0.45 μm syringe filter, and administered to rats (n=3) intraveneously at 5 mg/kg. CBD concentration (mg/mL) was measured at a series of timepoints as shown in FIG. 3A.

### 45. Pharmacokinetics Following Oral Administration

The formulation prepared in Example 16 was diluted to 10 mg/ml in 10 mM phosphate-buffered saline, filtered with an 0.45 μm syringe filter, and administered to rats (n=3) orally at 10 mg/kg. CBD concentration (mg/mL) was measured at a series of timepoints as shown in FIG. 3B. The calculated Cmax was 184 ng/mL and the calculated AUC was 447 ng·hr/mL.

### INCORPORATION BY REFERENCE

The contents of all cited references (including literature references, patents, patent applications, and websites) that may be cited throughout this application are hereby expressly incorporated by reference in their entirety for any purpose. In the event that any material incorporated by reference conflicts with the express content of this disclosure, the express content controls.

### Equivalents

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

What is claimed is:

1. An aqueous composition comprising cannabidiol (CBD), a first surfactant, and a second surfactant, wherein the first surfactant and the second surfactant are different.

2. The composition of claim 1, wherein the weight ratio of CBD to the first surfactant in the composition is from about 1:5 to about 5:1.

3. The composition of claim 1 or claim 2, wherein the weight ratio of the first surfactant to the second surfactant in the composition is from about 5:1 to about 1:5.

4. The composition of any one of claims 1 to 3, wherein the weight ratio of CBD to the second surfactant in the composition is from about 1:5 to about 5:1.

5. The composition of any one of the preceding claims, wherein the concentration of CBD in the composition is from about 0.01% to about 15% CBD w/v (g/mL).

6. The composition of claim 5, wherein the concentration of CBD in the composition is from about 0.01% to 0.5%, 0.5% to 1.5%, 1.5% to 2.5%, 2.5% to 3.5%, 3.5% to 4.5%, 4.5% to 5.5%, 5.5% to 6.5%, 6.5% to 7.5%, 7.5% to 8.5%, 8.5% to 9.5%, 9.5% to 10.5%, 10.5% to 11.5%, 11.5% to 12.5%, 12.5% to 13.5%, 13.5% to 14.5%, or 14.5% to 15%.

7. The composition of any one of the preceding claims, wherein the first surfactant is a hydrophilic, non-ionic surfactant.

8. The composition of claim 7, wherein the first surfactant is poloxamer 407 or a combination of surfactants comprising poloxamer 407, optionally wherein the combination further comprises poloxamer 188.

9. The composition of claim 7, wherein the first surfactant is or comprises poloxamer 338.

10. The composition of any one of the preceding claims, wherein the second surfactant is a hydrophilic, non-ionic surfactant.

11. The composition of claim 10, wherein the second surfactant comprises a PEGylated castor oil, a PEGylated hydrogenated castor oil, a polyoxyethylene ester of a hydroxylated long-chain, saturated fatty acid, or a polyoxyl castor oil, or a combination thereof.

12. The composition of claim 10 or 11, wherein the second surfactant comprises a polysorbate, optionally wherein the polysorbate is polysorbate 80.

13. The composition of any one of claims 10-12, wherein the second surfactant comprises PEG 35 castor oil.

14. The composition of any one of claims 10-13, wherein the second surfactant comprises Solutol HS-15.

15. The composition of any one of claims 10-14, wherein the second surfactant comprises d-α-tocopheryl polyethylene glycol 1000 succinate.

16. The composition of any one of claims 10-15, wherein the second surfactant comprises PEG 40 Hydrogenated Castor Oil.

17. The composition of any one of the preceding claims, wherein the composition is an aqueous micellar solution.

18. The composition of claim 17, wherein the Z-average particle size is less than about 50 nm, less than about 40 nm, less than about 30 nm, less than about 20 nm, or less than about 10 nm.

19. The composition of any one of the preceding claims, which is free of organic solvents, and/or wherein the composition is a pharmaceutical composition.

20. The composition of any one of the preceding claims, wherein the concentration of the first surfactant is 2-10 wt %.

21. The composition of any one of the preceding claims, wherein the concentration of the second surfactant is 5-15 wt %.

22. The composition of any one of the preceding claims, wherein the weight ratio of CBD to the first surfactant is in the range of 1:2 to 3:1.

23. The composition of any one of the preceding claims, wherein the weight ratio of CBD to the second surfactant is in the range of 1:2 to 2:1.

24. The composition of any one of the preceding claims, further comprising a buffer.

25. The composition of claim 24, wherein the buffer is a citrate buffer or a phosphate buffer.

26. The composition of any one of the preceding claims, having a pH in the range of 3-7, optionally wherein the pH is in the range of 3-6, 3-5, 3.5-4.5, or 3.75-4.25, or wherein the pH is about 4.

27. A method of making the aqueous composition of any one of claims 1 to 25, comprising:
forming a first solution comprising CBD, a first surfactant, water, and an organic solvent;
removing the organic solvent from the first solution to form a second solution; and
adding a second surfactant to the second solution to obtain the aqueous composition.

28. The method of claim 27, wherein removing the organic solvent comprises distillation.

29. The method of claim 27 or 28, wherein removing the organic solvent comprises rotary evaporation.

30. The method of any one of claims claim 27-29, wherein the organic solvent is or comprises ethanol, optionally wherein the ethanol is present in the first solution in a concentration of 15% to 60% by weight.

31. The method of claim 30, wherein the organic solvent is or comprises one or more of an alcohol, alkane, ether, ester, or ketone.

32. The method of claim 30, wherein the organic solvent is or comprises one or more of ethanol, isopropanol, pentane, ethyl ether, acetone, or ethyl acetate.

33. A method of making the aqueous composition of any one of claims 1 to 32, comprising:
forming a first mixture comprising CBD and a first surfactant as solids;
melting the CBD and the first surfactant; and
combining the first mixture with a solution comprising a second surfactant to obtain the aqueous composition.

34. The method of claim 33, wherein melting the CBD and the first surfactant comprises heating the CBD and the first surfactant to a temperature at or above 66° C., optionally wherein the temperature is in the range of 66° C.-100° C., 66° C.-90° C., 66° C.-80° C., 66° C.-75° C., or the temperature is about 70° C.

35. The method of claim 33 or 34, further comprising cooling the first mixture after melting the CBD and the first surfactant, e.g., to room temperature or a temperature in the range of 18° C.-40° C.

36. The method of claim 33 or 34, wherein the temperature of the first mixture is at or above 40° C., 45° C., 50° C., 55° C., 60° C., 66° C., 68° C., or 70° C. when combined with the solution comprising the second surfactant.

37. The method of claim 36, wherein the temperature of the first mixture when combined with the solution comprising the second surfactant is in the range of 66° C.-100° C., 66° C.-90° C., 66° C.-80° C., 66° C.-75° C., or the temperature is about 70° C.

38. The method of any one of claims 33-37, further comprising adding water to the first mixture before melting.

39. The method of any one of claims 33-37, wherein water is not added to the first mixture before melting.

40. The method of any one of claims 33-39, wherein organic solvent is not added to the first mixture before melting.

41. The method of any one of claims 27-40, further comprising filtering the CBD concentrate.

42. The method of any one of claims 27-41, comprising diluting the CBD concentrate with a diluent to form a CBD diluted concentrate.

43. The method of any one of claims 27-42, wherein at least one of the aqueous compositions, the CBD diluted concentrate, the first solution, or the second solution is a solution.

44. The method of claim 43, wherein at least one of the CBD concentrate, CBD diluted concentrate, first solution, or second solution is a micellar solution.

45. A method of treating a condition, disease, or disorder in a subject in need thereof, comprising administering to the subject a therapeutically effective amount of the composition of any one of claims 1 to 26.

46. A method of treating a condition, disease, or disorder in a subject in need thereof, comprising diluting the composition of any one of claims 1 to 26 in a pharmaceutically acceptable diluent, thereby forming a diluted composition, and administering a therapeutically effective amount of the diluted composition to the subject.

47. The composition of any one of claims 1 to 26, for use in treating a condition, disease, or disorder.

48. The method of claim 45 or 46 or the composition for use of claim 46, wherein the disease, disorder, or symptom is pain (such as neuropathic pain or cancer-related pain), spasticity, anxiety, cognition, a movement disorder, epilepsy (such as childhood epilepsy, such as Lennox-Gastaut syndrome or Dravet syndrome), or dementia (such as Alzheimer's disease, Vascular Dementia, Dementia with Lewy bodies (DLB), Parkinson's disease, Frontotemporal dementia or Huntington's disease).

49. The method or composition for use of any one of claims 45-48, wherein the composition is a CBD diluted concentrate.

50. A composition comprising water, a surfactant, an organic solvent, and CBD, wherein the CBD is dissolved in the composition.

51. The composition of claim 50, wherein the surfactant is a hydrophilic, non-ionic surfactant.

52. The composition of claim 50 or 51, wherein the surfactant comprises one or more of poloxamer 407 and poloxamer 188.

53. The composition of any one of claims 50-52, wherein the surfactant comprises poloxamer 407.

54. The composition of any one of claims 50-53, wherein the surfactant comprises poloxamer 188.

55. The composition of any one of claims 50-54, wherein the organic solvent is ethanol.

56. The composition of any one of claims 50-55, wherein the organic solvent is butane.

57. The composition of any one of claims 50-56, wherein the organic solvent comprises one or more of an alcohol, alkane (such as a C₃, C₄, C₅, or C₆ alkane, such as butane or pentane), ether, ester, ketone, or any combination thereof, optionally wherein the organic solvent comprises ethanol, isopropanol, pentane, ethyl ether, acetone, ethyl acetate, or any combination thereof.

58. The composition of any one of claims 50-57, wherein the organic solvent is present in a concentration of 30-40 wt %, 40-50 wt %, 50-60 wt %, or 60-70 wt %.

59. The composition of any one of claims 50-58, wherein the concentration of CBD in the composition is 5-15 wt %, such as 5-6 wt %, 6-7 wt %, 7-8 wt %, 8-9 wt %, 9-10 wt %, 10-11 wt %, 11-12 wt %, 12-13 wt %, 13-14 wt %, or 14-15 wt %.

60. The composition of any one of claims 50-59, wherein the concentration of the surfactant in the composition is 4-10 wt %, such as 4-5 wt %, 5-6 wt %, 6-7 wt %, 7-8 wt %, 8-9 wt %, or 9-10 wt %.

61. A method of preparing a solvent-free composition comprising CBD, the method comprising removing the organic solvent from the composition of any one of claims 50-60.

62. The method of claim 61, wherein removing the organic solvent comprises removal by distillation.
